(19) United States
(12) Patent Application Publication
(10) Pub. No.: US 2026/0035756 A1
(43) Pub. Date: Feb. 5, 2026

(54) BIOLOGICAL TEST SAMPLING KIT

(71) Applicant: HEMODX AS, Oslo (NO)

(72) Inventor: Praveen SHARMA, Oslo (NO)

(73) Assignee: HEMODX AS, Oslo (NO)

(21) Appl. No.: 19/099,002

(22) PCT Filed: Aug. 9, 2023
(86) PCT No.: PCT/EP2023/072116

§ 371 Date: Jan. 27, 2025

(30) Foreign Application Priority Data
Aug. 9, 2022 (GB) 2211645.3

(51) Int. Cl. C12Q 1/70; C12Q 1/6806; C12Q 1/6883; G01N 33/68

(52) U.S. Cl. CPC C12Q 1/701; C12Q 1/6806; C12Q 1/6883; G01N 33/6896; C12Q 2600/156; G01N 2800/2821; G01N 2800/2835

(57) ABSTRACT

The present invention relates to methods and kits for the collection, preservation and storage of analytes of interest present in biological test samples (biofluid samples). In particular, the present invention relates to means to collect the biological test sample such that substantially all pathogen present in the biological test sample is inactivated, while preserving the analyte of interest in a format to allow subsequent analysis.

### FIELD OF INVENTION

The present invention relates to collection devices, methods and kits for the collection, preservation and storage of analytes of interest present in biological test samples (biofluid samples). In particular, the present invention relates to means to collect the biological test sample such that substantially all pathogen present in the biological test sample is inactivated, while preserving the analyte of interest in a format to allow subsequent analysis.

### BACKGROUND

Biological test sample collection is an integral component of clinical medicine and research. Access to high-quality biological test samples, collected and handled in standardized ways to minimise potential bias or confounding factors, is key to laboratory testing process.

The pre-analytical phase of laboratory testing involves steps such as sample collection, transportation, sample preparation, and storage, and can account for up to 70% of errors during the total diagnostic process.

Additionally, some biological test sampling methods require cold chain—constant cold storage between the obtaining and testing of samples. This can add a significant cost to testing, and can prevent the use of such methods where suitable cold chain is not available.

As such, there is a need for biological test sampling devices that permit simple sample collection, transportation, sample preparation, and storage, ideally devices that permit increased automation and simpler processing.

Furthermore, the COVID-19 pandemic highlighted the urgent need for pre-analytical methodologies that present minimal risk of pathogen transmission between patients, health workers and laboratory staff, whilst ensuring that the biological samples are of a sufficient quality for accurate, reliable testing.

In the pre-analytical stage for routine confirmation of cases of COVID-19, collecting the proper respiratory tract specimen at the right time from the right anatomic site was essential for a prompt and accurate molecular diagnosis. Additionally, regular testing was required to minimise disease transmission—highlighting the need for methods that ensure patient compliance.

Although nasopharyngeal swabs are commonly used in respiratory virus diagnostics, collecting them causes discomfort to patients due to the procedure's invasiveness, limiting compliance for repeat testing. It also presents a considerable risk to healthcare workers administering the nasopharyngeal swabs, as it can induce patients to sneeze or cough, expelling pathogen onto the healthcare worker.

As such, a more reliable and less resource-intensive sample collection method is urgently needed; ideally one that accommodates self-collection (e.g. in the home or settings convenient to users such as the work place), is non-invasive, easy to self-administer and does not require cold chain storage/transport.

Furthermore, a sample collection method that accommodates self-collection permits routine testing for conditions or diseases in a pre-symptomatic or asymptomatic state—such as cancers, and dementia diseases such as Alzheimer's disease or Parkinson's disease. This is especially relevant where the self-collection method permits ambient transport of the sample(s) to centralised labs for analysis. Such routine testing can result in early clinical/therapeutic intervention and thus provide the best possible outcome to patients.

For instance, the neuropathologic markers of Alzheimer's disease are believed to manifest ten to fifteen years before evident cognitive symptoms. However, there are currently no routine tests for pre-symptomatic Alzheimer's disease.

Instead, Alzheimer's disease is currently diagnosed using a battery of costly and invasive investigations, in addition to clinical evaluation, cognitive tests and MR-imaging to exclude other diseases—this is not conducive to routine testing. For instance, the laboratory-based tests include testing cerebrospinal fluid (CSF) for key biomarkers of Alzheimer's disease, such as amyloid beta (Aβ), total tau and phosphorylated tau. However, testing CSF is not ideal as it requires lumbar punctures—these are dangerous, invasive and can only be administered by trained professionals.

There is a particular need for such routine, pre-symptomatic testing for Alzheimer's disease, given that current medications for Alzheimer's disease are known to delay the onset and/or slow the progression of symptoms.

Additionally, routine testing via a non-invasive sample collection method that accommodates self-collection can be used as an initial cost effective step, prior to applying further more costly diagnostic tests (i.e. a funneling/screening test). This has the potential to ensure that only relevant patients move further down the relevant diagnostic path, which reduces medical costs.

As saliva sampling is non-invasive and easy to self-administer, it emerged as an appealing alternative to nasopharyngeal swabs. For instance, studies indicate that SARS-CoV-2 can be detected from the saliva of COVID-19 patients at sensitivity levels comparable to nasopharyngeal or oropharyngeal sampling.

Saliva sampling collection devices for subsequent nucleic acid analysis are known, for instance, U.S. Pat. No. 5,939,259 or US patent application 2013/0289265, which are incorporated herein by reference.

However, there remains a need for saliva sampling devices that provide simple, convenient, safe and reliable pre-analytics, whilst both minimising risk of pathogen transmission and permitting the use of subsequent high quality laboratory testing—such as Nucleic Acid Amplification Tests (NAAT) like real-time reverse transcription-PCR (RT-PCR) assays.

### SUMMARY OF THE INVENTION

There is provided a biological test sampling device and kit for easy sampling of a biofluid such as saliva from a subject. Suitably the collection device further allows the sampled biofluid to be transported cheaply and safely to analytical laboratories for detection and analysis.

Suitably the collection of a biofluid such as saliva allows analysis of analytes of interest (biomarkers) such as nucleic acids and/or proteins present in the biofluid to be undertaken. Analysis includes the identification and/or quantification of the analyte of interest.

The term nucleic acid refers to all forms of RNA, for example mRNA, miRNA, rRNA, tRNA, piRNA, NcRNA, and/or DNA, for example genomic DNA, recombinant RNA and/or DNA and analogues of RNA or DNA including nucleotide analogues. Suitably the nucleic acid may be single stranded or double stranded.

According to a first aspect of the invention there is provided a collection device for the collection and storage of an analyte of interest from a biofluid sample, wherein the biofluid sample is suspected of comprising the analyte of interest and comprises one or more pathogens,
wherein the collection device comprises a solid matrix incorporating an inactivating solution adapted to inactivate substantially all of the one or more pathogens present in the biofluid sample and preserve at least a portion of the analyte of interest in a format that permits subsequent analysis,
wherein the inactivating solution comprises a combination of protein denaturants selected from one or more detergents, one or more chaotropic salts, one or more weak bases, one or more chelators and one or more reducing agents,
wherein in use, when the biofluid sample is provided to the solid matrix, substantially all of the one or more pathogens in the biofluid sample is inactivated and at least a portion of any analyte of interest present in the biofluid sample is preserved in a format that permits subsequent analysis.

This can be highly advantageous; by using an inactivating solution that is adapted to inactivate substantially all of the one or more pathogens while preserving at least a portion of the analyte of interest in a format that permits subsequent analysis (e.g. identification and/or quantification), the collection device minimises the risk of pathogen transmission to healthcare and laboratory personnel, whilst permitting the use of subsequent high quality laboratory testing of the analyte of interest.

It will be understood that the inactivating solution may be adapted such that it inactivates substantially all of the one or more pathogens over an inactivation period. Suitably, the inactivating solution may be adapted such that when a biofluid sample is provided to the solid matrix, substantially all of the pathogen in the biofluid sample is inactivated within 45 minutes of the biofluid sample being provided to the collection device. Preferably, within 30 minutes of the biofluid sample being provided to the collection device. More preferably, within 15 minutes of the biofluid sample being provided to the collection device. In particular, the inactivation period may be decreased by increasing the concentration and/or strength of the one or more chaotropic salts, or the concentration and/or strength one or more detergents, or a combination thereof.

Suitably, the inactivation solution may be adapted such that it inactivates substantially all of the pathogen in the biofluid sample, such that at least 90%, at least 95%, at least 99%, at least 99.9 or at least 99.99% of the infective microorganisms present in the one or more pathogens are inactivated. Preferably, the inactivation solution is adapted to inactivate substantially all of the one or more pathogens present in the biofluid sample such that no infective microorganisms (e.g. virions) can be detected.

As used herein, the term “pathogen” is defined as being a bacterium, virus, or other microorganism that can cause disease, and is considered synonymous for “infective microorganism”.

Suitably, the pathogen is at a high titre. As used herein, the term “high titre” refers to the quantity of infective pathogen in a sample that is significantly above (≥2×, ≥5×, ≥10×, or ≥50×) the minimum infective dose to cause an infection of that pathogen in 50% of a population of animals susceptible to infection by that pathogen. In humans, this is known as the Human ID50 or HID50.

The term “biofluid” refers to a sample, either in liquid or solid form, suspected of having dissolved, suspended, mixed or otherwise contained therein, any analytes of interest, for example, nucleic acids, proteins or metabolites.

The term “biofluid” also refers to a sample of whole blood, plasma, serum, saliva, lymph, synovial fluid, cerebrospinal cord fluid, semen, saliva, urine, faeces, sputum, vaginal lavage, fluid from infection lesions, or the like of humans or animals, physiological and pathological body liquids, such as secretions, excretions, exudates and transudates, any cells or cell components of humans, animals, plants, bacterial, fungi, plasmids, viruses, parasites, or the like that contain the analyte of interest, and any combination thereof.

As used herein, “suspected of comprising” is defined as having the potential to comprise. Suitably, the biofluid sample comprises the analyte of interest. Thus, subsequent analysis of the analyte of interest can include where the analyte of interest is detected and/or quantified. Suitably, either the biofluid sample does not comprise the analyte of interest, or its concentration is so low that it is below detection levels. Thus, subsequent analysis of the analyte of interest can include where the analyte of interest is not detected and/or quantified.

Suitably, the analyte of interest may be a component of at least one of the pathogens present in the biofluid sample. Suitably, the analyte of interest may be a component of the biofluid sample other than the pathogen.

Suitably, the analyte of interest may be a molecule in the sample that can be analysed: determined/identified or measured/quantified. For example, nucleic acids, polynucleotides, oligonucleotides, proteins, polypeptides, oligopeptides, enzymes, amino acids, receptors, carbohydrates, fatty acids, vitamins, minerals, metabolites, lipids, hormones, cells, any intra- or extra-cellular molecules and fragments, virus, viral molecules and fragments. Preferably, the analyte of interest is a nucleic acid, or a peptide/protein. More preferably, the analyte of interest is a nucleic acid.

Suitably the analyte of interest may be a nucleic acid, including either or both DNA or RNA. As used herein, the term “nucleic acid” or “polynucleotide” refers to RNA or DNA that is linear or branched, single or double stranded, a hybrid, or a fragment thereof. The term also encompasses RNA/DNA hybrids.

Suitably the analyte of interest may be a protein, polypeptide, oligopeptide or peptide. Preferably, where the protein, polypeptide, oligopeptide or peptide can aggregate in solution and/or is hydrophobic. More preferably, where the protein, polypeptide, oligopeptide or peptide is prone to aggregate in solution and is hydrophobic.

As disclosed in Freeman et al., “State of the Science in Dried Blood Spots”, Clinical Chemistry, Volume 64, Issue 4, 1 Apr. 2018, Pages 656-679, Amado et al. “One decade of salivary proteomics: Current approaches and outstanding challenges”, Clinical Biochemistry 46, 506-517 (2013), Yan et al., “Systematic comparison of the human saliva and plasma proteomes”, Proteomics—Clinical Applications, 3, 116-134 (2009) and Pawlik et al., “The Role of Salivary Biomarkers in the Early Diagnosis of Alzheimer's Disease and Parkinson's Disease”, Diagnostics 2021, 11, 371, which are herein incorporated by reference, there are believed to be more than 2000 analytes in blood or saliva that may be used for diagnostic and/or prognostic purposes. Saliva has been identified as a functional equivalent to serum. For example, one proteomic study found 1939 proteins in saliva, with 597 proteins that have been observed in plasma.

As mentioned, the neuropathologic markers of Alzheimer's disease are believed to manifest ten to fifteen years before cognitive symptoms emerge. As such, reliable, simple and self-administered tests for biomarkers of Alzheimer's disease would be conducive for routine screening of the disease significantly prior to the emergence of cognitive symptoms. Such routine screening would permit early therapeutic intervention, in order to minimise and/or delay the onset of symptoms. This is also thought to be the case for Parkinson's disease and other neurodegenerative diseases.

As mentioned in Pawlik et al., “The Role of Salivary Biomarkers in the Early Diagnosis of Alzheimer's Disease and Parkinson's Disease”, Diagnostics 2021, 11, 371, a number of biomarkers for Alzheimer's Disease and Parkinson's Disease are present in saliva. For instance, concentrations of Aβ protein and peptide fragments involved in the amyloid cascade are present in saliva are significantly elevated in those affected by Alzheimer's—this contrasts with the reduced concentration of Aβ protein observed in the spinal fluid of such individuals.

Without wishing to be bound by theory, it is believed that elevated concentrations of Aβ₁₋₄₂ in a patient's saliva is indicative of Alzheimer's disease. Preferably, where the Aβ₁₋₄₂ is one or more of the following:
Aβ protein,
Aβ-42,
Aβ-40,
Aβ-34, and/or
Aβ-20.

Suitably, where a patient's saliva has a concentration of one or more of the above Aβ₁₋₄₂ proteins that is at or above 1.5× the average concentration of that found in the saliva of those without Alzheimer's disease (the negative control), it is considered indicative of Alzheimer's disease. Preferably, the concentration of the one or more Aβ₁₋₄₂ proteins is 1.5-3× the concentration of the negative control, more preferably about 2.45× the concentration of the negative control.

Notably, Aβ-42 and Aβ-40 are involved in amyloidosis and are insoluble/hydrophobic. Conversely, Aβ-34 and Aβ-20 are involved in amyloid clearance and are soluble.

Additionally or alternatively, while the total tau protein concentration is not altered in the saliva of those affected by Alzheimer's, the ratio of salivary phosphorylated tau (p-tau) concentration to total salivary tau protein (t-tau) concentration is indicative of Alzheimer's disease and/or frontotemporal dementia. Suitably, a salivary p-tau:t-tau ratio more than 1.2× that of the average concentration of that found in the saliva of those without Alzheimer's disease (the negative control), is considered indicative of Alzheimer's disease and/or frontotemporal dementia. Preferably, the p-tau is one of the following:
a) The S400, T403 and S404 residues,
b) The S396 residue, or
c) The S404 residue.

More preferably, the p-tau is at the S396 residue.

Further suitable biomarkers for Alzheimer's disease that can be detected in saliva include glial fibrillary acidic protein, lactoferrin and neuronal damage marker, NFL.

For instance, a salivary lactoferrin concentration below 7.43 μg/mL is considered indicative of either Alzheimer's disease or amnestic mild cognitive impairment.

Preferably, a test for Alzheimer's disease using a collection device of the present invention would assay biofluid samples for a combination of the above neuropathologic markers, which would improve accuracy.

Suitably, the analyte of interest may be preserved on the collection device for as short as the time necessary to transfer a sample of biofluid or a portion thereof from a collection source to the place where subsequent analysis is to be performed. Suitably preservation may occur for a period of several minutes, hours, days, months or greater.

The temperature conditions under which a biological specimen may be stored in the collection device provided by the present invention are not limited. Typically, samples are shipped and/or stored at ambient or room temperature, for example, from about 10° C. to about 50° C., preferably about 15° C. to about 25° C. Suitably, the samples may be stored in a cool environment. For example, in short-term storage, the samples can be refrigerated at about 2° C. to about 10° C. In yet another example, the samples may be refrigerated at about 4° C. to about 8° C. In another example, in long-term storage, the samples can be frozen at about −80° C. to about −10° C. In yet another example, the samples can be frozen from about −50° C. to about −20° C. In addition, the collection device is preferably stored in dry or desiccated conditions and/or under an inert atmosphere.

Suitably, multiple types of biofluid may be collected by the collection device. For example faecal and saliva samples from the same subject.

Suitably, the collection device may be able to be used to collect more than one biofluid type. This can be highly advantageous to allow more accurate or sensitive testing or to allow the development of infection to be monitored. For example, in the recent COVID-19 pandemic, nasopharyngeal and oropharyngeal swabs were the recommended specimen types for Covid-19 diagnostic testing. Antibody response testing, using plasma and serum and ELISA based assays for the detection of IgM/IgG antibodies may also be utilised and dried blood spots may be suitably used for such ELISA testing. Moreover, a combination of saliva and another biofluid may be used, for example saliva and faecal matter may be used to monitor the progression of a disease, for example COVID-19 in a patient.

Advantageously, the collection device allows for the collection of multiple dried biofluid specimens in a home setting. Further advantageously, the collection device can be used to conveniently collect more than one type of biofluid specimen from a subject/patient whilst posing no or reduced biohazard risk for any transmission of infection between patients and health workers.

Suitably the solid matrix may be an absorbent and/or adsorbent material that does not bind irreversibly to nucleic acids. Suitably the solid matrix may comprise cellulosic material, porous glasses, woven porous polymers, non-porous polymers or combinations thereof. Suitably, the solid matrix may be a non-dissolvable matrix, for example cellulose, in particular SF cellulose, capable of receiving and retaining a saliva sample comprising nucleic acid. Such solid matrices are also known as “filter paper”. Suitably the solid matrix may be a porous planar or sheet material.

It is understood that the solid matrix (particularly non-dissolvable filter solid matrices) have an ability to adsorb and/or absorb a biofluid readily and quickly, as well as release the analyte of interest efficiently and precisely. In preferred embodiments, each solid matrix can adsorb and/or absorb at least 0.05 ml, 0.1 ml, 0.15 ml, or preferable 0.2 ml or greater, of biofluids suspected of containing an analyte of interest.

The collection device may include one or more designated areas, each composed of a solid matrix configured to receive and adsorb and/or absorb a biofluid sample. Where a plurality of designated areas is provided, this allows for obtaining more than one type of biofluid specimens while avoiding any cross contamination during sample collection storage and transport. These may be colour coded to indicate to the user the biofluid to be provided to each area. Suitably, different designated areas may comprise different components. For example, a particular designated area may comprise means to aid the drying of a particular biofluid. Additionally or alternatively, a particular designated area may include means to remove or bind a component of a biofluid placed onto the area.

Suitably, each of the one or more designated areas may comprise a matrix holding portion, for example a card frame or polystyrene or plastics or back/supporting layer. Each matrix holding portion may substantially surround and/or support a solid matrix to retain the solid matrix in a fixed position relative to the remainder of the collection device. Suitably, each of the one or more designated areas may comprise a portion to encase or cover its solid matrix after collection of the sample. Suitably the portion to encase or cover the solid matrix may be a sleeve portion. Suitably the portion to encase or cover the solid matrix may be one or more flaps that can be moved from a first position to at least a second position to alter access to the solid matrix.

Suitably a “back layer” or “support layer” may be formed from a suitable substrate, for example a polymeric or plastics material, paper, plastic, metal foil, laminate comprising metal foil, metallized film, glass, silicon oxide coated films, and aluminium oxide coated films, liquid crystal polymer layers, and layers of nano-composites, metal or metal alloys and acrylic. Suitably, any non-porous material may be used to form a back layer.

Suitably, the collection device comprises a back layer comprising one or more apertures and a middle layer, where each aperture is covered by a solid matrix attached to the back layer. Where the collection device comprises multiple apertures, each covered by a solid matrix, the solid matrices are separated from each other, in order to avoid cross contamination among different biofluid types.

Suitably, where the collection device comprises the back and middle layer, the collection device may further comprise a printable front layer (top layer) with a replica of the one or more apertures in the back layer. The front layer is fixed, for example glued on top of the middle layer so that the one or more apertures align, such that the solid matrix or matrices are exposed to air on both the front and back sides. Materials for construction of the front layer are not limited, but can be paper, plastic, metal foil, laminate comprising metal foil, metallized film, glass, silicon oxide coated films, and aluminium oxide coated films, liquid crystal polymer layers, and layers of nano-composites, metal or metal alloys and acrylic. In the preferred embodiments, the front layer is made of paper suited for printing and lamination. Important information can be printed on the front side of the card.

Suitably, the one or more detergents may be selected from a group comprising or consisting ionic detergents such as sodium dodecyl sulphate (SDS), deoxycholate, cholate and sarkosyl, and non-ionic detergents such as the Triton family (octoxinol, e.g. Triton X100, Triton X-114), Nonidet P-40 (NP-40), Igepal® CA-630 and the Tween family (e.g. Tween-20 and Tween-80).

Suitably, the one or more chaotropic salts may be selected from a group comprising or consisting guanidium salts (e.g. guanidium isothiocyanate (GITC), guanidine thiocyanate, guanidine hydrochloride), sodium iodide, sodium perchlorate, sodium thiocyanate and potassium iodide.

The one or more weak bases are included for pH buffering purposes and may be selected from a group comprising or consisting 2-Amino 2-hydroxymethyl-propane-1,3-diol (Tris), 2-(N-morpholino) ethanesulfonic acid (MES), 3-(N-morpholino)propane sulfonic acid (MOPS), citrate buffers, 4-(2-hydroxyethyl)-1-piperazineethanesulfonic acid (HEPES), and phosphate buffers. It is considered that a person skilled in art would recognize that the pH of the buffer selected would typically be in the range of 3 to 8.

The one or more chelators are included to bind the divalent metal ions magnesium and calcium, as well as to bind transition metal ions, particularly iron. Suitably, the one or more chelators may be selected from a group comprising or consisting ethylenediaminetetraacetic acid (EDTA), Ethyleneglycoltetraacetic acid (EGTA) and 8-Hydroxyquinoline (8HQ).

Suitably, the one or more reducing agents may be selected from a group comprising or consisting dithiothreitol (DTT) dithioerythritol (DTE), L-glutathione (GSH) and Tris (2-carboxyethyl phosphine hydrochloride (TCEP) and 2-mercaptoethanol (2-ME).

Where the solid matrix comprises cellulose (e.g. filter paper), the solid matrix incorporating inactivating solution may comprise at least 50 wt % cellulose and 12-40 wt % of the one or more chaotropic salts. Without wishing to be bound by theory, it is felt that this concentration of chaotropic salts is effective in inactivating substantially all of the pathogen in the biofluid sample, while preserving the analyte of interest in the biofluid sample in a format that permits the subsequent identification and/or quantification.

Where the solid matrix comprises cellulose (e.g. filter paper), the solid matrix incorporating inactivating solution may comprise at least 50 wt % cellulose, 12 to 40 wt % of the one or more chaotropic salts, 1 to 5 wt % of the one or more detergents, 0.5 to 1.5 wt % of the one or more weak bases, 0.1 to 0.6 wt % of the one or more chelators, and 0.2 to 0.7 wt % of the one or more reducing agents. Without wishing to be bound by theory, it is felt that, this combination of components within these ranges are especially effective in inactivating substantially all of the pathogen in the biofluid sample, while preserving the analyte of interest in the biofluid sample in a format that permits the subsequent identification and/or quantification.

Suitably, the inactivating solution comprises or consists of guanidinium isothiocyanate (GITC) (also known as thiocyanic acid with guanidine (1:1)), sodium dodecyl sulphate (SDS), octoxinol (also known as Triton X-100), Tris base (also known as trometamol), ethylenediaminetetraacetic acid (EDTA), and dithiothreitol (also known as DL-1,4-dithiothreitol). Without wishing to be bound by theory, it is felt that, this combination of components within these ranges are exceptionally effective in inactivating substantially all of the pathogen in the biofluid sample, while preserving the analyte of interest in the biofluid sample in a format that permits the subsequent identification and/or quantification.

Where the solid matrix comprises cellulose (e.g. filter paper), the solid matrix incorporating inactivating solution may comprise at least 50 wt % cellulose, in the range 12 to 40 wt % thiocyanic acid with guanidine (1:1), 1 to 3 wt % sodium dodecyl sulphate, 0.5 to 2 wt % octoxinol, 0.5 to 1.5 wt % trometamol, 0.1 to 0.6 wt % ethylenediaminetetraacetic acid, and 0.2 to 0.7 wt % DL-1,4-Dithiothreitol.

Suitably, the inactivating solution may lyse cells and denature some or all proteins whilst protecting analytes of interest from degradation, such as by protecting nucleic acids from within lysed cells from nucleases. This allows nucleic acids to be preserved for future detection and analysis, whilst effectively inactivating substantially all of the pathogen in the biofluid sample.

Suitably, where the analyte of interest is a nucleic acid, it can be released after collection and storage such that it can be amplified by conventional techniques such as polymerase chain reaction (PCR) or other techniques using DNA and/or RNA.

Suitably, the inactivating solution may lyse cells and denature proteins whilst protecting other protein or peptides (analytes of interest) from degradation and aggregation. This allows protein or peptides to be preserved for future detection and analysis, whilst effectively inactivating substantially all of the pathogen in the biofluid sample.

Suitably, where the analyte of interest is a protein or a metabolite, it can be released after collection and storage such that it can be detected by conventional techniques such as enzyme-linked immunosorbent assay (ELISA), mass spectrometry or other techniques that detect proteins or metabolites. As the inactivation solution can denature proteins, for any techniques that rely on antibodies, it is preferable that the antibodies have binding specificity to exposed epitopes when the protein/peptide analyte of interest is denatured. Where such antibodies are not available, other techniques such as mass spectrometry is suitable. Additionally or alternatively, the protein/peptide analyte of interest may be reconstituted in solution by incubation in a buffer comprising solubilisation agents such as CHAPS, for instance a PBS buffer comprising 0.45% CHAPS.

Once a biofluid sample is absorbed and/or adsorbed on to the solid matrix and dried, the collection device or part thereof can be safely sent for analysis using standard postal systems at ambient temperatures and a wide range of humidity level. Suitably, any analytes of interest present on the solid matrix can then be either eluted or resolublised off the solid matrix.

Suitably, where the analyte of interest is a component of at least one of the pathogens, the collection device may be used to test for the presence, absence or quantity of viral or bacterial agents, in particular viral or bacterial pathogens. Suitably, viral agents may be selected from corona virus, influenza virus, norovirus, rabies (lyssavirus), Human papillomavirus, Epstein-Barr virus, Herpes simplex virus, Hepatitis virus, in particular Hepatitis C virus, Monkeypox virus and HIV.

Suitably, where the analyte of interest is a component of at least one of the pathogens, the collection device may be used to test for the presence, absence or quantity of bacterial agents for example in relation to microbiome profiling or identification, bacterial dysbiosis, periodontitis, dental carries, diabetes, obesity, metabolic disorder, cancer, CVD, immuno-related systemic diseases.

Suitably, wherein the analyte of interest is a biomarker for a neurodegenerative disease, the collection device may be used in the diagnosis of that neurodegenerative disease. Preferably, the diagnosis is an initial screening/filtering process, prior to more extensive and/or reliable diagnostic methods. Preferably, the neurodegenerative disease is selected from a group consisting Alzheimer's disease, Parkinson's disease, other memory disorders, Huntington's disease, motor neuron disease, multiple system atrophy and progressive supranuclear palsy. More preferably, the neurodegenerative disease is either Alzheimer's disease or Parkinson's disease.

Suitably, the inactivating solution is able to inactivate a high titre of SARS CoV 2 virions effectively, with no detectable virions within 45 minutes of providing the biofluid sample to the collection device.

Suitably, RNA can be recovered from SAR CoV 2 virus dried on the solid matrix and used for RT PCR detection of viral RNA.

Suitably, the collection device may comprise a sample receiving portion, an identification tag portion, and a cover portion. Suitably, the identification portion and at least part of the cover portion can be folded to cover the sample receiving portion.

Suitably at least part of identification portion can be removed and retained by the user. Suitably the sample receiving portion may comprising an identification tag, for example a barcode.

Suitably the collection device may comprise a card frame with a solid matrix portion composed of a filter paper pre-treated, with inactivating solution to inactivate viral pathogen and stabilize RNA for a downstream analytical procedure.

Suitably the collection device may comprise a card frame with a solid matrix portion composed of a filter paper pre-treated to inactivate viral and/or bacterial pathogen and stabilize RNA for a downstream analytical procedure. Suitably the collection device may be pre-treated to stabilize DNA for a downstream analytical procedure.

Suitably the collection device may comprise at least two flaps that may be moved from a first position to a second position wherein in a first position the filter paper is accessible to provide a sample to the filter paper and in a second position the filter paper is not accessible to provide a sample to the filter. Suitably text information for a user may be provided on the collection device. Suitably the collection device may include a unique device identification (UDI) number. Suitably the collection device may include at least two copies of a unique device identification (UDI) number wherein in a first copy of the unique device identification (UDI) number is removable from the collection device. For example, the first copy may be provided on a first portion of the collection device that, via a frangible attachment is joined to a second portion of a collection device.

Suitably the sample receiving portion comprising a dye, for example Alizarin Red S, which shows the presence of saliva and the spreading of saliva in the sample receiving portion. Suitably the dye shows the drying process of the saliva into the collection device. Suitably the dye may be provided in the collection device in the range 0.01 to 0.05%.

Suitably recovering analyte for testing from the collection device may be undertaken using extraction kits from third party vendors, for example Qiagen. Suitably recovery may be undertaken using Trizol LS, QIAamp Viral RNA Mini Kit and suitable buffers. Suitably detecting may be undertaken in accordance with known clinical tests.

Suitably RNA recovery from the solid matrix may be undertaken using Trizol RNA extraction

For example, a Trizol RNA extraction method and downstream RT PCR may be undertaken using techniques as discussed in Corman V M, Landt O, Kaiser M, Molenkamp R, Meijer A, Chu D K, et al. Detection of 2019 novel coronavirus (2019-nCoV) by real-time RT-PCR. Euro Surveill. 2020; 25, which is herein incorporated by reference.

Suitably two 4.5 mm discs, or portions in the range of about 2 mm to 8 mm in length and breadth, of the solid matrix may be transferred to an Eppendorf tube containing Trizol LS (375 μL) and OptiPro (125 μL). For extraction, samples may be agitated on a shaker and RNA extracted with chloroform. RNA may then be purified with the RNA Clean and Concentrator-5 kit according to the manufacturer's instructions and eluted in nuclease-free water. Eluted RNA can be used for downstream qPCR purposes as exemplified below.

Suitably recovering analyte for testing from the collection device may be undertaken using a method of any one of the second, third or fourth aspects of the invention.

According to a second aspect of the invention, there is provided a method to detect nucleic acid provided from a dried biofluid from a collection device, Wherein the collection device comprises a solid matrix, for example filter paper, which was pre-treated with an inactivating solution adapted to inactivate substantially all of the one or more pathogens present in the biofluid sample and preserve at least a portion of the analyte of interest in a format that permits subsequent analysis, wherein the inactivating solution comprises a combination of protein denaturants selected from one or more detergents, one or more chaotropic salts, one or more weak bases, one or more chelators and one or more reducing agents, and Wherein the method comprises the steps:
washing a portion of the solid matrix comprising dried the dried biofluid with prewash buffer at room temperature to form a previously dried biofluid, wherein the prewash buffer comprises
   60%-80% ethanol, and
   inactivating solution comprising a combination of protein denaturants selected from one or more detergents, one or more chaotropic salts, one or more weak bases, one or more chelators and one or more reducing agents,
washing a portion of the solid matrix comprising the previously dried biofluid with a first wash solution, wherein the first wash solution comprises at least 70% ethanol,
optionally washing a portion of the solid matrix comprising the previously dried biofluid with a second wash solution at room temp, wherein the second wash solution is at least 95% ethanol, suitably to reduce the drying time of the preserved nucleic acids on the solid matrix
eluting analyte of interest from the solid matrix by incubating the solid matrix RNAase free water.

Suitably, the collection device is a collection device of the first aspect.

Suitably, the dried biofluid was previously a biofluid sample suspected of comprising the analyte of interest and comprised pathogen.

Suitably, the dried biofluid was produced by providing the biofluid sample to the solid matrix, wherein substantially all of the pathogen in the biofluid sample was inactivated and at least a portion of any analyte of interest present in the biofluid sample was preserved in a format that permits subsequent analysis; the biofluid sample was then dried to provide the dried biofluid.

Suitably the method allows extraction of nucleic acids, such as RNA, DNA and RNA/DNA.

Without wishing to be bound by theory, it is believed that the prewash step helps to solubilize dried components and remove protein aggregates and cell debris from the sample. The first wash step is believed to remove salts and impurities that may affect analyte detection techniques such as polymerase chain reaction or other nucleic acid detection techniques. A second wash step is believed to aid the drying of the sample

Suitably, the prewash solution comprises
about 60%-80% ethanol, suitably about 70% ethanol, and
inactivating solution, wherein the inactivating solution comprises a combination of protein denaturants selected from one or more detergents, one or more chaotropic salts, one or more weak bases, one or more chelators and one or more reducing agents.

Suitably, the one or more detergents may be selected from a group consisting ionic detergents such as sodium dodecyl sulphate (SDS), deoxycholate, cholate and sarkosyl, and non ionic detergents such as the Triton family (octoxinol, e.g. Triton X100, Triton X-114), Nonidet P-40 (NP-40), Igepal® CA-630 and the Tween family (e.g. Tween-20 and Tween-80).

Suitably, the one or more chaotropic salts may be selected from a group consisting guanidium salts (e.g. guanidium isothiocyanate (GITC), guanidine thiocyanate, guanidine hydrochloride), sodium iodide, sodium perchlorate, sodium thiocyanate and potassium iodide.

The one or more weak bases are included for pH buffering purposes and may be selected from a group consisting 2-Amino 2-hydroxymethyl-propane-1,3-diol (Tris), 2-(N-morpholino) ethanesulfonic acid (MES), 3-(N-morpholino)propane sulfonic acid (MOPS), citrate buffers, 4-(2-hydroxyethyl)-1-piperazineethanesulfonic acid (HEPES), and phosphate buffers. It is considered that a person skilled in art would recognize that the pH of the buffer selected would typically be in the range of 3 to 8.

The one or more chelators are included to bind the divalent metal ions magnesium and calcium, as well as to bind transition metal ions, particularly iron. Suitably, the one or more chelators may be selected from a group consisting ethylenediaminetetraacetic acid (EDTA), Ethyleneglycoltetraacetic acid (EGTA) and 8-Hydroxyquinoline (8HQ).

Suitably, the one or more reducing agents may be selected from a group consisting dithiothreitol (DTT) dithioerythritol (DTE), L-glutathione (GSH) and Tris (2-carboxyethyl phosphine hydrochloride (TCEP) and 2-mercaptoethanol (2-ME).

Where the solid matrix comprises cellulose (e.g. filter paper), the solid matrix incorporating inactivating solution may comprise at least 50 wt % cellulose and 12-40 wt % of the one or more chaotropic salts.

Where the solid matrix comprises cellulose (e.g. filter paper), the solid matrix incorporating inactivating solution may comprise at least 50 wt % cellulose, 12 to 40 wt % of the one or more chaotropic salts, 1 to 5 wt % of the one or more detergents, 0.5 to 1.5 wt % of the one or more weak bases, 0.1 to 0.6 wt % of the chelators, and 0.2 to 0.7 wt % of the reducing agents.

Suitably, the inactivating solution comprises or consists of guanidinium isothiocyanate (GITC), sodium dodecyl sulphate (SDS), octoxinol, Tris base, ethylenediaminetetraacetic acid (EDTA), and dithiothreitol.

Suitably wherein the inactivating solution comprises or consists of thiocyanic acid with guanidine (1:1), sodium dodecyl sulphate, octoxinol, trometamol, ethylenediaminetetraacetic acid, and DL-1,4-Dithiothreitol. Suitably in the range 12 to 40% thiocyanic acid with guanidine (1:1), 1 to 3% sodium dodecyl sulphate, 0.5 to 2% octoxinol, 0.5 to 1.5% trometamol, 0.1 to 0.6% ethylenediaminetetraacetic acid, and 0.2 to 0.7% DL-1,4-Dithiothreitol.

Suitably the prewash solution is about 70% ethanol and 10% to 20% RNAase free water and 10%-20% inactivating solution.

Suitably the first wash solution is about 70% ethanol and 30% RNAase free water. Suitably the second wash solution is about 95% ethanol.

The volume of washing buffers will depend on the size of matrix that needs to be treated. For example if two 4.5 mm discs have been punched out from the solid matrix and subjected to washing prior to nucleic acid elution around 400 μl will be used. If one 4.5 mm disc is used, the volume of prewash solution could be reduced to 200 μl. The amount of analyte present in the biospecimen that is to be measured will dictate the amount of matrix required to be utilised for example one, two or three discs. Depending on the biospecimen to be analysed, 2× washing may be optimal, 3× washing may be optimal, or even 4× or more may be required. The protocol below is optimized for both saliva and faecal sample. For only saliva a 2× washing step has been shown to work well. A washing time of 2 min is preferred as it reduces the time of each cycle; however, a washing time of 5 min may be used. Gentle shaking is advantageous to allow solutions come evenly in contact with the solid matrix. For drying, 10 min-30 min at ambient conditions is typically sufficient. To eluate RNA, room temperature is preferred, but the temperature may be increased to 50 degrees (10-30 min or more). If only DNA is to be eluted, the temperature may be increased up to 95 degrees C. (10-30 min or more). Note, RNA will degrade at 95 degrees and hence a temperature at or greater than 95 degrees C. is not optimal for RNA elution. To eluate both RNA and DNA, 10-50 degrees is preferred. The elution time will depend upon the temperature. Room temperature may require longer elution time.

Suitably in specific embodiments, a dedicated specimen extraction area or multiple areas of the solid matrix may be removed and washed as follows:
Wash 3× with 400 μl of prewash buffer at room temp. Incubate for 2 min, shaking 450 rpm, each washing step,
Wash 3× with 200 μl of the first wash solution. Incubate for 2 min, shaking 450 rpm, each washing step,
Wash 3× with 100 μl of the second wash solution at room temp. Incubate for 2 min, shaking 450 rpm, each washing step.

Suitably each wash step may be undertaken multiple times, for example two or three times. Suitably the solid matrix may be incubated between wash steps, suitably the solid matrix may be washed with shaking, for example at 450 rpm at any wash steps or all was steps.

Suitably the washed matrix may be dried at room temperature for 30 minutes prior to elution of nucleic acid from the solid matrix by incubating the solid matrix in 100 μl RNAase free water at 50° C. for 60 min. in a water bath.

Advantageously the method the present invention may be undertaken without the need for centrifugation. This may be particularly advantageous where automation of sample handling is desired. Suitably the method of the invention may be undertaken more quickly than conventional methods such as Trizol extraction.

Suitably the biofluid may be saliva that has dried into the solid matrix, for example filter paper. Suitably the biofluid may be faecal matter that has dried into the solid matrix, for example filter paper. Suitably the biofluid may be a combination of saliva and biofluid that have been provided to different areas of the collection device. Suitably the biofluid may be a fluid from a lesion.

Further optionally, the method may comprise recovering nucleic acid from the saliva sample provided by the collection device. Suitably the method may further comprise detecting the nucleic acid provided in the saliva sample.

Suitably the method uses RT PCR detection of SARS CoV 2 viral RNA as known in the art. Suitably, nucleic acids can be recovered from collection device of the present invention (e.g a filter paper portion of the solid matrix comprising dried saliva) using a QIAamp Viral RNA kit (Qiagen) according to the manufacturer's instructions. Suitably, SARS CoV-2 detection may be undertaken using any suitable nucleic acid detection technique. For example by qPCR using E-Serbeco assay and LightCycler® 96 System.

Suitably the reaction set up uses TaqMan™ Fast Virus 1-Step Master Mix, E_Sarbeco_F (Seq ID No 1: 5′-ACAGGTACGTTAATAGTTAATAGCGT-3′), E_Sarbeco_R (Seq ID No 2: 5′-ATATTGCAGCAGTACGCACACA-3′) E_Sarbeco_P (Seq ID No 3: FAM-5′-ACACTAGCCATCCTTACTGCGCTTCG-3′-BHQ1) and input total RNA. A standard panel with Twist Synthetic SARS-CoV-2 RNA control ranging from 101 to 105 RNA copies/mL can be used to generate a standard curve. Thermal cycling can be performed at 55° C. for 10 min for reverse transcription, followed by 95° C. for 3 min and then 45 cycles of 95° C. for 15 s and 58° C. for 30 s using the LightCycler® 96 System. RNA titers (copies/mL) can be calculated by interpolation of cycle threshold (Ct) values in the standard curve generated from the standard panel, using the LightCycler® software.

Suitably, a portion of the solid matrix and an aliquot of buffer may be incubated together at room temperature for 50° C. for 30 min. Suitably, the incubated portion of solid matrix and aliquot of buffer can be mixed with ethanol (96-100%). Suitably the solid matrix portion and buffer mixed with ethanol may be centrifuged. Suitably the washed matrix and solution may be transferred to a QIAamp Mini column or the like. Suitably RNA eluate from the column is used in a RT-PCR reaction or stored at −20° C. until further use.

Suitably the analytical sensitivity of the method to detect nucleic acid from a dried sample provided by the collection device is comparable to that of a wet sample.

According to a third aspect of the invention, there is provided a method to detect an analyte of interest provided from a dried biofluid, from a collection device, where the analyte of interest is a protein, a polypeptide, an oligopeptide or a peptide,
Wherein the collection device comprises a solid matrix, for example filter paper, which was pre-treated with an inactivating solution adapted to inactivate substantially all of the one or more pathogens present in the biofluid sample and preserve at least a portion of the analyte of interest in a format that permits subsequent analysis, wherein the inactivating solution comprises a combination of protein denaturants selected from one or more detergents, one or more chaotropic salts, one or more weak bases, one or more chelators and one or more reducing agents, and
Wherein the method comprises the steps:
   eluting the analyte of interest from the solid matrix by incubating at least a portion of the solid matrix comprising the dried biofluid with an elution buffer, then removing the elution buffer, wherein the elution buffer comprises
      one or more buffering salts,
      one or more solubilisation agents and
      one or more protease inhibitors.

Suitably, the collection device is a collection device of the first aspect.

Suitably, the dried biofluid was previously a biofluid sample suspected of comprising the analyte of interest and comprised pathogen.

Suitably, the dried biofluid was produced by providing the biofluid sample to the solid matrix, wherein substantially all of the pathogen in the biofluid sample was inactivated and at least a portion of any analyte of interest present in the biofluid sample was preserved in a format that permits subsequent analysis; the biofluid sample was then dried to provide the dried biofluid.

Suitably, the method may further comprise briefly washing the solid matrix using a pre-elution buffer, prior to eluting the analyte of interest from the solid matrix. The pre-elution buffer may or may not have the same composition as the elution buffer, but comprises:
one or more buffering salts,
one or more solubilisation agents and
one or more protease inhibitors.

The wash with the pre-elution buffer is provided to wet the solid matrix and remove any particulates. Suitably, the washing may comprise providing the solid matrix with the pre-elution buffer, this mixture is then briefly vortexed, microcentrifuged and incubated at room temperature for 5 min with agitation—shaking at 5500 rpm in a tabletop shaker in a tabletop shaker. The supernatant is then aspirated, to remove any particulates from the solid matrix.

Suitably, the elution may comprise providing the solid matrix with elution buffer, and incubating the mixture at 37° C. for 1 hour with agitation—shaking at 5500 rpm in a tabletop shaker in a tabletop shaker. This mixture is then briefly vortexed, microcentrifuged and the supernatant aspirated to provide a solution comprising the analyte of interest, wherein the analyte of interest is a protein, a polypeptide, an oligopeptide and/or a peptide.

Preferably, the protein, polypeptide, oligopeptide or peptide can aggregate in solution and/or is hydrophobic. More preferably, where the protein, polypeptide, oligopeptide or peptide is prone to aggregate in solution and is hydrophobic.

Where the concentration of peptide/protein in saliva is low (e.g. in the order of pg/μL concentrations), a suitable amount of analyte may be collected by providing more solid matrix and/or dried biofluid. For instance, where 100 μL of saliva is provided to a collection device of the present invention, five 6 mm diameter discs of solid matrix (which equate to ˜45 μL of the saliva) are sufficient to elute Aβ-42 and/or Aβ-40 at concentrations suitable for ELISA assays. The amount of buffer used to elute the analyte of interest from the solid matrix is also relevant as this affects the final concentration. In the above example, ˜150 μL of elution buffer is suitable to elute Aβ-42 and/or Aβ-40 at concentrations suitable for ELISA assays.
Suitably, the elution buffer comprises
buffering salts to provide a pH of 6-9 preferably between 6.5 and 8.5,
0.1-0.6% of the one or more solubilisation agents and
1-3% of the protease inhibitors.

Suitably, the buffering salts may be phosphate buffered saline (PBS) or Tris-buffered saline (TBS). Preferably, the buffering salts are PBS at pH of ˜7.4,

Suitably, the one or more solubilisation agents may be selected from a group consisting 3-[(3-cholamidopropyl)dimethylammonio]-1-propanesulfonate (CHAPS), 3-([3-Cholamidopropyl]dimethylammonio)-2-hydroxy-1-propanesulfonate (CHAPSO), and/or thioflavin (such as thioflavin T or thioflavin S). Preferably, the solubilisation agents are CHAPS and/or thioflavin S.

CHAPS and CHAPSO are non-denaturing zwitterionic detergents used to solubilize biological macromolecules such as proteins, particularly where those biological macromolecules are sparingly soluble or insoluble in aqueous solution due to their native hydrophobicity.

Thioflavin prevents protein/peptide aggregation, particularly amyloid aggregation, where it binds to amyloid fibrils but not amyloid monomers.

Suitably, the protease inhibitors may be selected from a group consisting PLAAC—protease inhibitors pepstatin, leupeptin, antipain, aprotinin, and chymostatin Roche Cat. No: 11836170001, Halt™ protease inhibitor cocktail, Thermo Scientific™, Pierce™ protease inhibitor cocktail, Thermo Scientific™, Halt™ protease and phosphatase inhibitor cocktail, Thermo Scientific™, Pierce™ protease and phosphatase inhibitor cocktail, Thermo Scientific™ and PMFS protease inhibitor, Thermo Scientific™.

Suitably, the elution buffer further comprises one or more of the following:
One or more chelating agents,
One or more carrier proteins,
One or more phosphatase inhibitors, and
One or more biocides,

Suitably, the one or more chelating agents may be selected from a group consisting ethylenediaminetetraacetic acid (EDTA), Ethyleneglycoltetraacetic acid (EGTA) and 8-Hydroxyquinoline (8HQ). Preferably the chelating agent is EDTA.

Suitably, the one or more biocides may be selected from a group consisting ProClin and sodium azide. Preferably, the biocide is ProClin.

Suitably, the one or more carrier proteins may be selected from a group consisting bovine serum albumen (BSA) and keyhold limpet hemocyanin (KLA). Preferably, the carrier protein is BSA.

In a preferred embodiment, the elution buffer comprises:
buffering salts to provide a pH of ˜7.4,
0.1-0.6% of the one or more solubilisation agents,
1-3% of the one or more protease inhibitors,
0.3-1.5% of the one or more chelating agents,
0.05-2% of the one or more carrier proteins and
0.03-0.07% of the one or more biocides,

In a further preferred embodiment, the elution buffer comprises:
PBS to provide a pH of 7.4
0.01-0.1% of one or more non-denaturing zwitterionic detergent(s),
0.1-0.5% Thioflavin,
1-3% of the one or more protease inhibitors,
0.3-1.5% of the one or more chelating agents,
0.05-2% of the one or more carrier proteins,
0.03-0.07% of the one or more biocide(s),

In a further preferred embodiment, the elution buffer comprises:
PBS to provide a pH of 7.4
0.01-0.1% CHAPS
0.1-0.5% Thioflavin S,
1-3% protease inhibitors,
0.3-1.5% EDTA,
0.05-2% BSA,
0.03-0.07% ProClin,

Suitably the method may further comprise detecting the analyte of interest. Suitable methods are known in the art, such as ELISA. However, the inactivation solution can denature proteins. Suitably, denatured proteins may be refolded using methods known in the art.

Where protein denaturing occurs, for any techniques that rely on antibodies, it is preferable that the antibodies have binding specificity to exposed epitopes when the protein/peptide analyte of interest is denatured. However, such antibodies may not be known. Thus, detection of the analyte of interest may require alternative means such as mass spectrometry.

According to a fourth aspect of the invention, there is provided a method to detect an analyte of interest provided from a dried biofluid from a collection device, where the analyte of interest is a protein, a polypeptide, an oligopeptide or a peptide,
wherein the collection device comprises a solid matrix, for example filter paper, which was pre-treated with an inactivating solution adapted to preserve at least a portion of the analyte of interest in a format that permits subsequent analysis,
wherein the inactivating solution comprises one or more protease inhibitors and one or more solublising agents and
Wherein the method comprises the steps:
   eluting the analyte of interest from the solid matrix by incubating at least a portion of the solid matrix comprising the dried biofluid with an elution buffer, then removing the elution buffer, wherein the elution buffer comprises
      one or more buffering salts,
      one or more solubilisation agents and
      one or more protease inhibitors.

Advantageously, this may be used to enable methods of detection that are otherwise affected by protein denaturation. For instance, it enables detection methods that rely on antibody techniques where the antibody has binding specificity to epitopes that are not exposed when the protein/peptide is denatured.

Suitably, this collection device may inactivate substantially all pathogen present in the biofluid sample when it dries to provide the dried biofluid.

Suitably, the inactivating solution does not comprise detergents or chaotropic salts. Otherwise, the statements that apply to the third aspect also apply to the fourth aspect.

According to a further aspect of the present invention, there is provided a collection device for the collection and storage of an analyte of interest from a biofluid sample,
wherein the biofluid sample is suspected of comprising an analyte of interest and comprises pathogen,
wherein the analyte of interest is a protein, a polypeptide, an oligopeptide or a peptide,
wherein the collection device comprises a solid matrix, for example filter paper, which was pre-treated with an inactivating solution adapted to preserve at least a portion of the analyte of interest in a format that permits subsequent analysis,
wherein the inactivating solution comprises one or more protease inhibitors and one or more solublising agents,
wherein in use, when the biofluid sample is provided to the solid matrix, at least a portion of any analyte of interest present in the biofluid sample is preserved in a format that permits subsequent analysis.

Suitably, this collection device may inactivate substantially all pathogen present in the biofluid sample when it dries.

Suitably, the inactivating solution does not comprise detergents or chaotropic salts. It will be appreciated that this will result in inactivating less pathogen than the collection device of the third aspect. Otherwise, the statements that apply to the first aspect also apply to this further aspect.

Suitably, the collection device of this further aspect is for use in the method of the fourth aspect.

According to a fifth aspect of the present invention there is provided a kit, comprising a collection device of the first aspect of the invention and at least one of:
a. A pipettor to drop the biofluid onto the collection device,
b. a biofluid collection cup,
c. a bag, for example a zip lock or biohazard marked bag,
d. desiccant, and
e. a return shipping envelope for storage and transport of dried biofluid samples to a designated laboratory for analysis.

Suitably a kit may also include at least one of:
a. an Instruction for Use manual to guide sample collection,
b. gloves,
c. towelette,
d. temperature strip,
e. cleaning material and
f. a container for disposal of the kit.

Suitably, the collection kit comprises a collection device that further comprises a sample identification tag, for example a barcode.

According to a sixth aspect of the present invention there is provide a method of using the collection device of the first aspect of the invention to undertake at least one of profiling the microbiome, genotyping purposes, diagnosing diseases, selecting therapy, and determining disease severity.

Saliva, blood or faecal samples can be obtained as per required. As will be appreciated, nucleic acid obtained using the collection device discussed herein might be used for various clinical applications.

Suitably the collection device of the first aspect of the invention may be used with the method of any one of the second, third or fourth aspects, to undertake at least one of diagnosing diseases, selecting therapy, and determining disease severity.

For example, the collection device and/or method of the invention may be used in relation to infectious diseases, different cancer types (breast, prostate, pancreas, lung, colon etc.) and neurodegenerative diseases such as Alzheimer's disease and other memory disorders, Parkinson disease, Huntington's disease, motor neuron disease, multiple system atrophy and progressive supranuclear palsy.

### BRIEF DESCRIPTION OF THE FIGURES

An embodiment of the present invention will now be described by way of example only with reference to the accompanying figures in which:

FIG. 1 is an illustration of top view of a collection device of the present invention showing a sample receiving portion (b), a cover portion (a), and an identification tag portion (c).

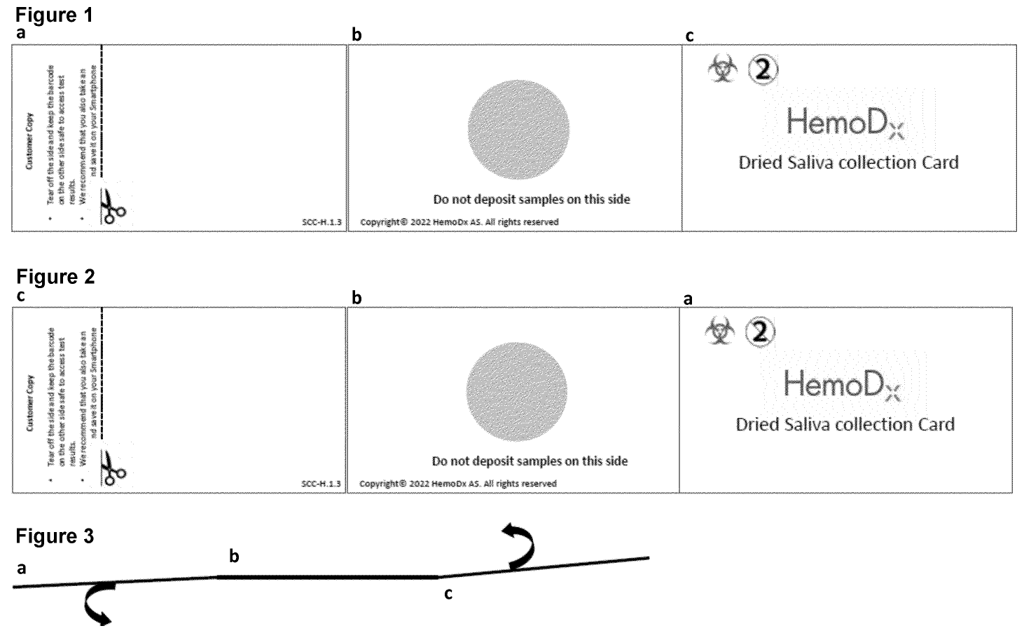

FIG. 2 is an illustration of a bottom view of a collection device of the present invention.

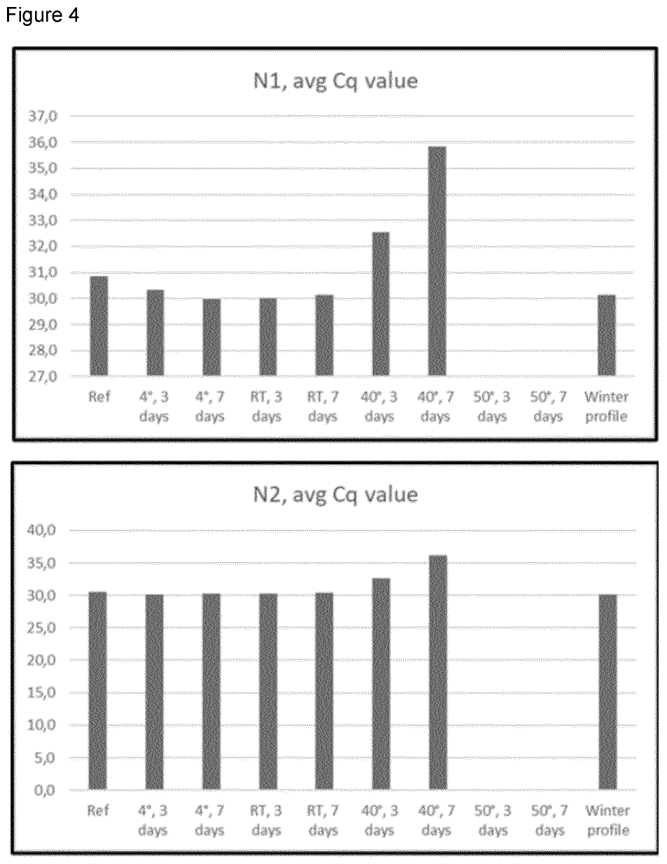

FIG. 3 is an illustration of the direction of folding of at least part of the identification portion (c) and a cover portion (a) when these are folded to cover the sample receiving portion (b).

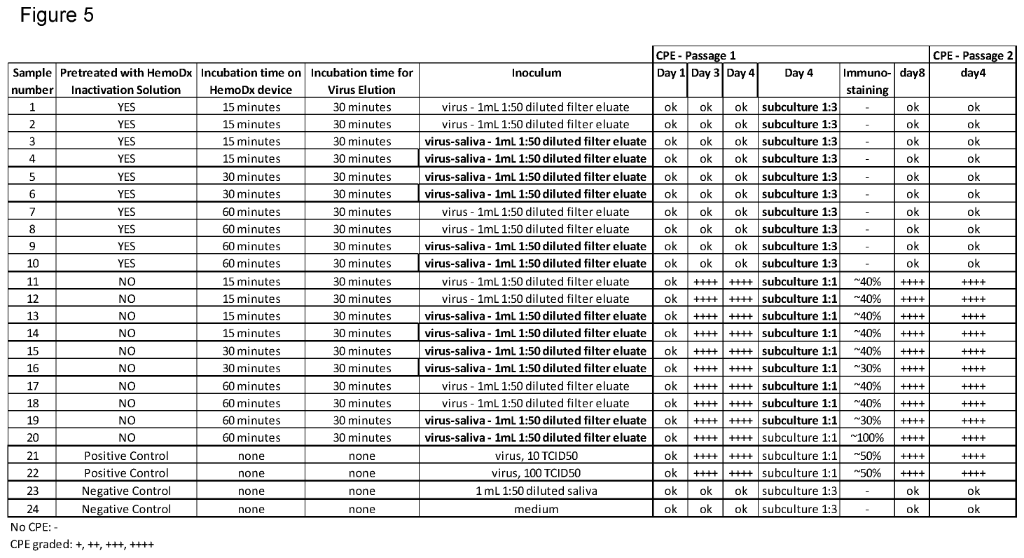

FIG. 4 is an illustration of analysis for the presence or absence of SARS CoV 2 viral RNA following an already established RT PCR procedure in an analytical laboratory using N1 and N2 assays.

FIG. 5 is an illustration of Table 1, and

FIG. 6 is an illustration of Table 2.

FIG. 7 illustrates average DI score per sample and preparation method with standard deviation bars.

FIG. 8 illustrates average abundance score per probe per preparation method in all samples.

FIG. 9 illustrates a schematic view of an unfolded (A) and folded (B) collection device.

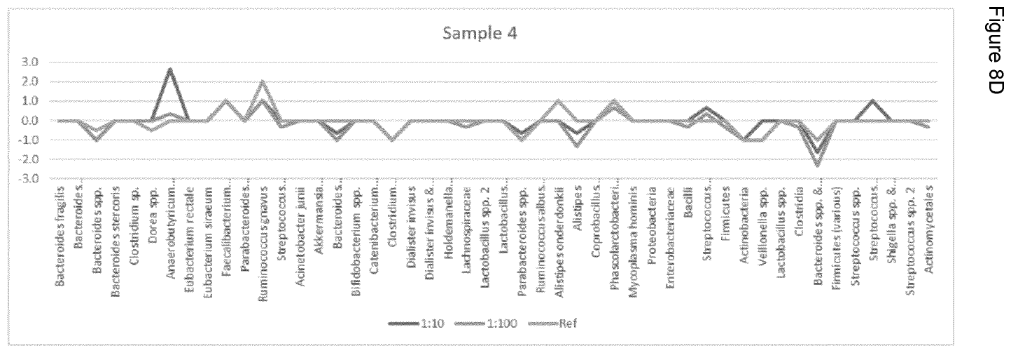

FIG. 10 illustrates a collection device with appropriate holes and apertures to distinguish between different biofluid types.

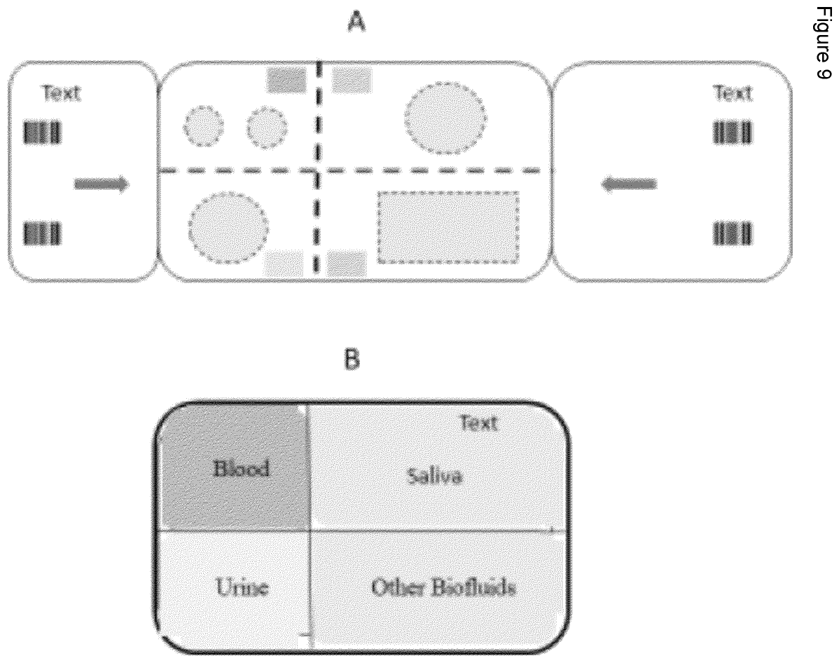

FIG. 11 illustrates filter paper solid matrices provided on (for example glued) onto a back or supporting layer of the biocollection device.

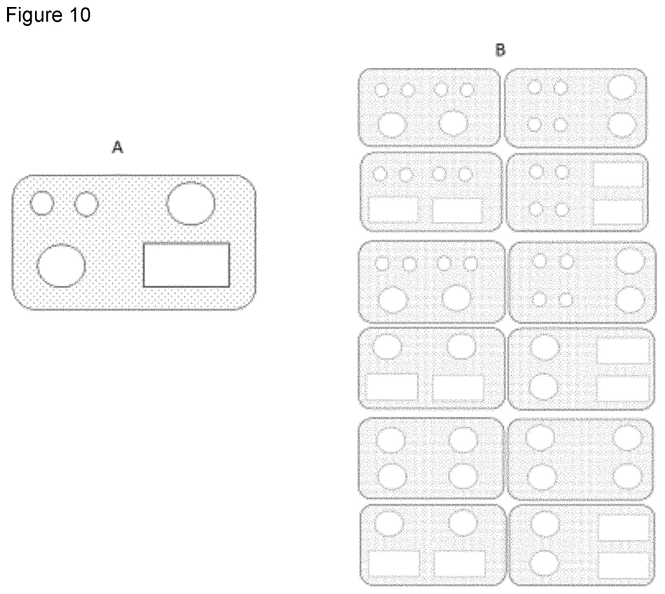

FIG. 12 shows the design of a preferred embodiment for collecting four different types of biofluid specimen. FIG. 12 A shows an example of back layer with appropriate holes and apertures. FIG. 12 B shows filter paper solid matrices glued on to the back layer to avoid cross contamination of different types of biofluid specimens. FIG. 12 C shows example of front layer with exact replica of holes and apertures as in FIG. 12A and flaps at the sides. FIG. 12 D shows an example of Front layer glued on top of the middle layer of the collection device.

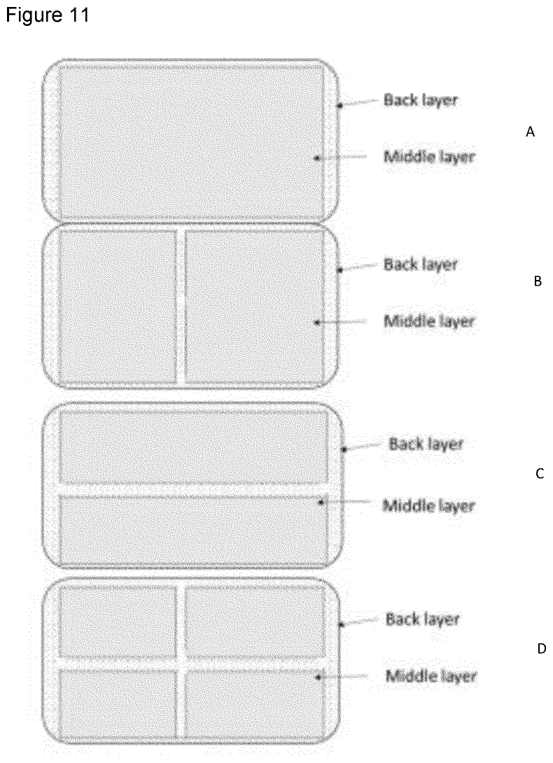

FIGS. 13 A, 13 B and 13 C show an example of an unprinted (13A), printed (13B) and folded (13C) collection device

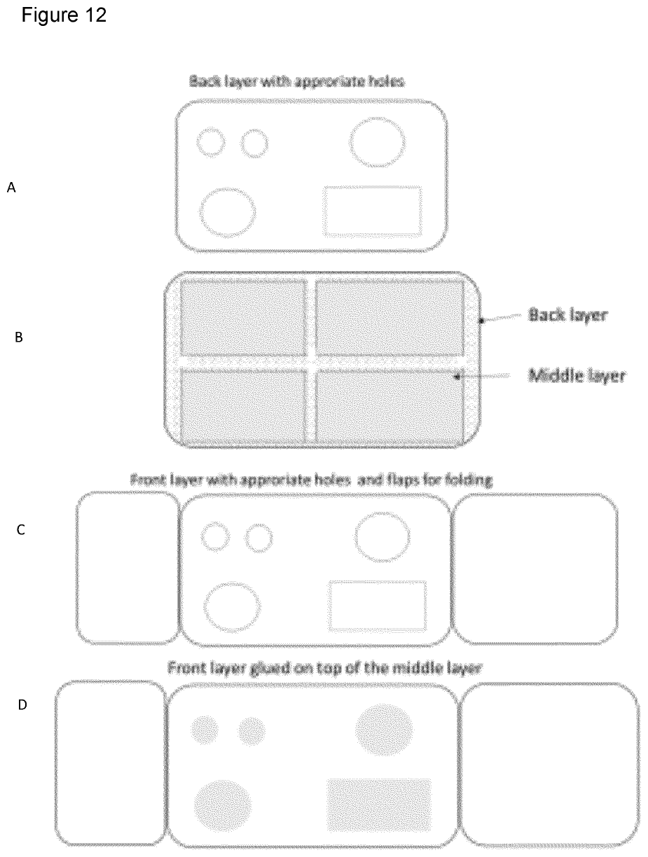

FIGS. 14 A and 14 B shows some designs of collection device indicating its flexibility for customization depending upon the application and purpose.

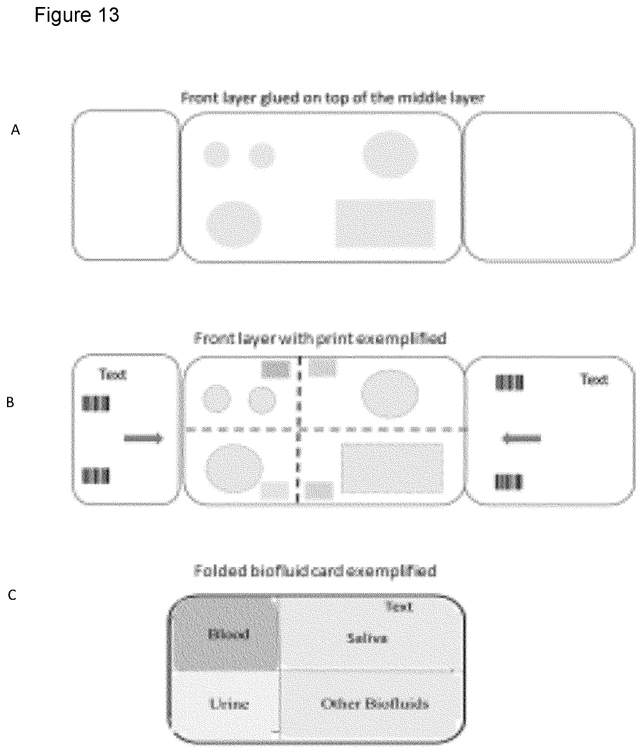

FIG. 15 A shows a schematic view of a collection device and 15 B shows drying of obtained biofluid on a flat surface following absorption of the biofluids on the collection device.

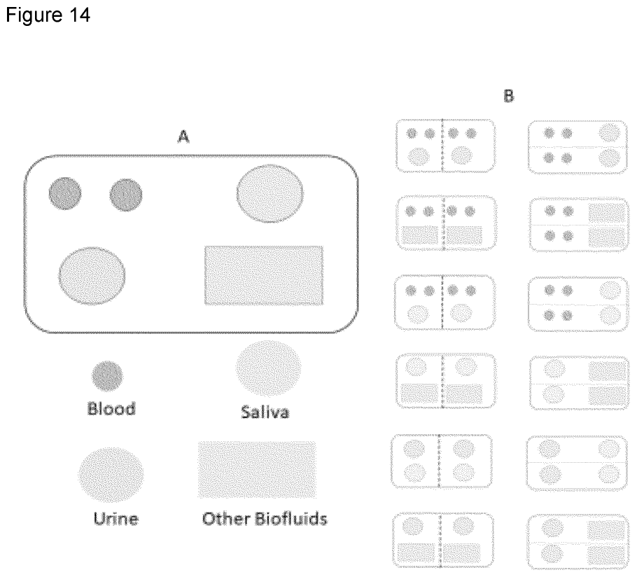

FIG. 16 is an illustration of the efficacy of amplification using Trizol RNA extraction (T), the washing and extraction method of the present invention (H), QiaGen Viral RNA extraction (Q).

FIG. 17 is an illustration of the viral RNA copy number extrapolated from Hvidovre dilution data using Trizol RNA extraction (T), the washing and extraction method of the present invention (H), QiaGen Viral RNA extraction (Q).

FIG. 18 is an illustration of the concentration of amyloid fragments Aβ-40 and Aβ-34 obtained from saliva samples using a collection device of the present invention, where their concentration was measured using two different analytical platforms: mesoscale (MSD) and SIMOA (SR-X).

### DETAILED DESCRIPTION

Saliva was collected by passive drooling and two drops of saliva was dropped onto the centre of a sample receiving portion of the collection device. The sample receiving portion comprises a filter paper with an indicator dye that clearly show the spread of saliva when saliva is dropped on it. Suitably a coloured, for example a pink coloured indicator dye that discolours to yellow/white with a red halo surrounding the area containing the dried saliva sample may be used. Once visually confirmed the collection device was air dried for at least 45 minutes. The card was then folded and placed into a biohazard marked aluminium bag containing desiccant. The aluminium bag was then placed inside a return envelope and sent to a designated laboratory for analysis. Each collection device includes a unique bar code identification for track and trace purposes. This unique bar code identification can be used to access results from testing reference laboratory.

### Example 1—Stability Studies

As background for the following examples: In the analytical stage, real-time reverse transcription-PCR (RT-PCR) assays remain the molecular test of choice for the etiologic diagnosis of pathogens such as SARS-CoV-2, while antibody-based techniques are used as supplemental tools. The viral genes targeted in such assays include the N, E, S and RdRP viral genes, among others. To avoid potential cross-reaction with other endemic coronaviruses as well as potential genetic drift of SARS-CoV-2, at least two molecular targets are included in an assay for routine confirmation.

Collection devices of the present invention were prepared by providing filter paper as a solid matrix. This was treated by immersing it in the following solution:

| Buffer Component | Concentration |
|---|---|
| GITC | 4M |
| SDS | 2% |
| Triton X-100 | 1% |
| Tris HCl (pH: 7.6) | 100 mM |
| EDTA | 10 mM |
| DTT | 20 mM |
| Alizharin red | 1 mM |

The filter papers was then dried to provide a solid matrix incorporating an inactivation solution within the following definition: at least 50 wt % cellulose, in the range 12 to 40 wt % thiocyanic acid with guanidine (1:1), 1 to 3 wt % sodium dodecyl sulphate, 0.5 to 2 wt % octoxinol, 0.5 to 1.5 wt % trometamol, 0.1 to 0.6 wt % ethylenediaminetetraacetic acid, and 0.2 to 0.7 wt % DL-1,4-Dithiothreitol.

This was then used in stability studies, which were undertaken under different temperature conditions during collection, drying, storage and transport to laboratories for testing. Stability of the collection device considering delayed transport time (seven days), normal postal time (three days), hot summer conditions (40 degrees C. and 50 degrees C.) and during winter profiles was undertaken.

A mixture of saliva and SARS CoV 2 viral stock dilution requiring 30-31 cycles of amplification was spotted on the filter paper and dried for 45 minutes at a BSL3 laboratory. The filter papers were sent to analytical laboratory for analysis in dry ice (−80 degrees C.). Triplicates of filter paper discs were incubated at 4 degrees C., room temperature, 40 degrees C. and 50 degrees C. for three days and seven days. For winter profile, the triplicates were incubated for 16 hours at −20 degrees C., followed by 4 hours at RT (20 degrees C.), followed by 2 hours at −20 degrees C., followed by 40 hours at 4 degrees C. and then 6 hours at 20 degrees C. Samples were analysed for the presence or absence of SARS CoV 2 viral RNA following RT PCR procedure already established in the analytical laboratory using N1 and N2 assays.

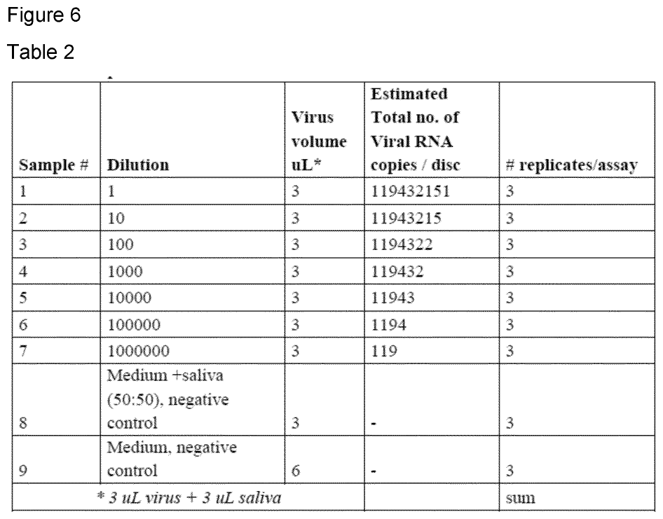

The results (FIG. 4) suggested that SARS CoV RNA on the collection device is highly stable when exposed to a winter profile. The RNA is also highly stable at both 4 degrees C. and room temp when incubated for 3 days and 7 days. An increased number of cycles was required when the collection device with RNA was incubated at 40 degrees C. for 3 days (2 cycles) and 7 days (6 cycles) suggesting instability of RNA at 40 degrees C. when subjected to very long time periods. No clear interpretable amplification was observed at 50 degrees C. suggesting that viral RNA was not stable at this temperature. Additional studies shall be performed to determine the stability of RNA at temperature range 40 degrees C. to 50 degrees C. to find how long RNA may be exposed at these temperatures.

### Example 2—Shelf-Life Stability

To determine the shelf life of the collection device of the present invention, collection devices were prepared in line with those of Example 1. These were then stored for 3, 6, 12 and 24-months and the assay of Example 1 was repeated, along with infectivity studies.

Inactivation of SARS-2 CoV2 was determined by viral activity—infectivity of eukaryotic cells by evaluating cytopathic effect (CPE) and S (spike) protein immunostaining.

Evaluation post three months based on CPE detection and immunological staining confirmed that the testing matrix retained its efficacy and completely inactivated the virus after 45 minutes contact time after three months of manufacturing.

Further, it was determined that detection of viral RNA by RT PCR was effective.

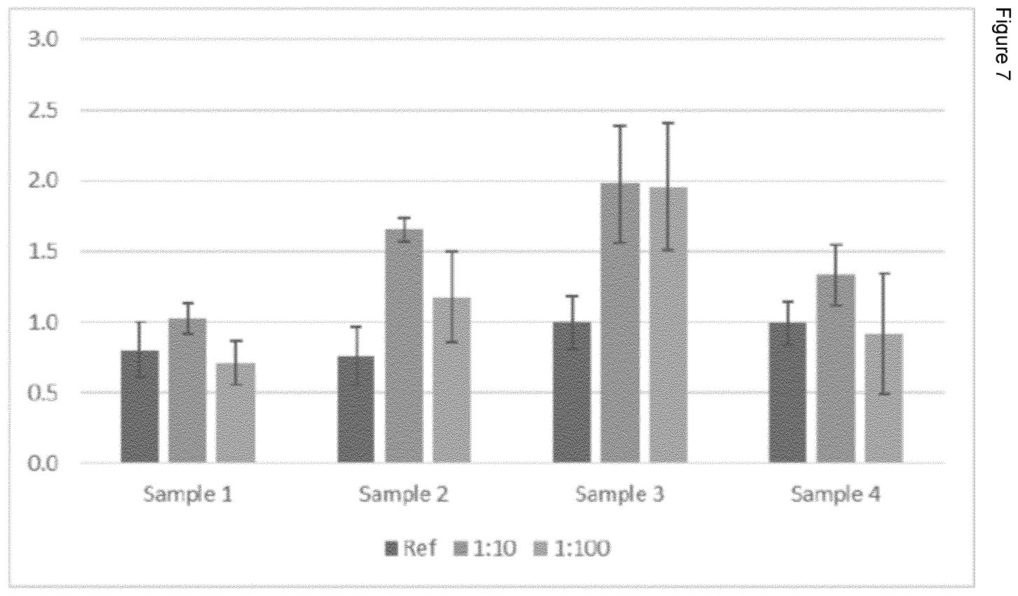

SARS-CoV 2 Virus stocks (serum-free media) originally derived from a Danish COVID-19 patient and subsequently propagated in Vero E6 cells were used (passage 39, 7.6 log 10 TCID50/ml. 50 μL virus stock or 50 μL 1:1 mix of saliva and virus stock were spotted onto filter paper discs (pretreated with inactivation solution and non treated) and incubated for 15 minutes, 30 minutes or 60 minutes. Filter eluate was obtained by filter incubation in 1 mL Vero E6 cell culture medium for 30 min incubation with subsequent shaking. 1 mL of 1:50 diluted filter eluate was transferred to cells (VeroE6, confluent T25 flask) and incubated for 30 minutes, subsequently, medium was added to each culture for a total volume of 4 mL. It was determined that at 1:50 dilution of filter paper eluate did not cause any CPE effect on cell cultures. Cultures were monitored in the microscope for CPE detection and by S protein immunostaining as summarized in the Table 1 (FIG. 5).

All cultures inoculated with filter paper pretreated with inactivation solution were negative for CPE and S staining, independent of incubation time (15, 30 and 60 min) and saliva spiking. All cultures inoculated with filter paper not pretreated with inactivation solution were positive for CPE and S staining, independent of incubation time (15, 30 and 60 min) and saliva spiking. A control culture inoculated with saliva alone did not develop CPE. Thus, filter paper including the inactivation solution completely inactivates SARS CoV 2 virus within 45 minutes of contact time (15 minutes on filter paper and 30 min Eluate incubation time).

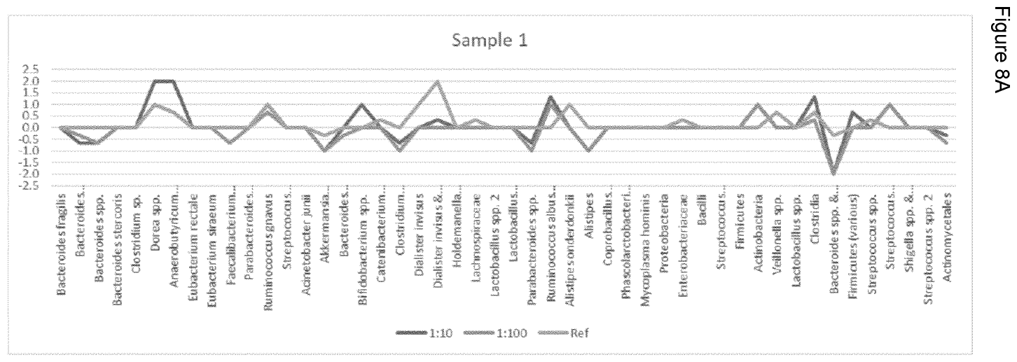

The analytical performance of the collection device was evaluated by spiking saliva with serial dilution of SARS CoV 2 viral stock 10.6 log 10 copies/mL and spotting on 6 mm pretreated filter paper discs (pretreated with Inactivation solution) in a BSL-3 facility (Table 2—FIG. 6). The filter paper discs containing the virus were dried for 45 minutes and sent to testing laboratories under conditions mimicking real life settings. Samples were be analysed for the presence or absence of SARS CoV 2 viral RNA following procedure already established in respective laboratories.

In a first test example condition filter paper discs containing virus were incubated in Trizol LS (375 μL) and OptiPro (125 μL) for 2 hours under agitation on a shaker at 250 RPM for 2 hours. RNA was extracted with chloroform in 5PRIME Phase Lock Gel Heavy tubes and purified with the RNA Clean and Concentrator-5 kit according to the manufacturer's instructions and eluted in 15 μL nuclease-free water.

For viral RNA detection the reaction was set up using TaqMan™ Fast Virus 1-Step Master Mix: 400 nM 1E_Sarbeco_F (Seq ID No: 1 5′-ACAGGTACGTTAATAGTTAATAGCGT-3′), 400 nM E_Sarbeco_R (Seq ID No: 2 5′-ATATTGCAGCAGTACGCACACA-3′) and 200 nM E_Sarbeco_P (Seq ID No: 3 FAM-5′-ACACTAGCCATCCTTACTGCGCTTCG-3′-BHQ1) and 2.5 μL input total RNA.

A standard panel with Twist Synthetic SARS-CoV-2 RNA control ranging from 101 to 105 RNA copies/mL was used to generate a standard curve. Thermal cycling was performed at 55° C. for 10 min for reverse transcription, followed by 95° C. for 3 min and then 45 cycles of 95° C. for 15 s and 58° C. for 30 s using the LightCycler® 96 System. The RNA titers (copies/mL) were calculated by interpolation of cycle threshold (Ct) values in the standard curve generated from the standard panel, using the LightCycler® software.

In a second test example condition, two different RNA recovery methods were tested. In one method, two 4.5 mm discs were washed as below:
Wash 3× with 200 μl of prewash buffer and at room temp. Incubate for 2 min, shaking 450 rpm, each washing step.
Wash 3× with 200 μl of a first wash solution containing 70% ethanol. Incubate for 2 min, shaking 450 rpm, each washing step.
Wash 3× with 100 μl of a second wash solution containing 95% ethanol at room temp. Incubate for 2 min, shaking 450 rpm, each washing step.

In another method, the RNA was recovered using QIAamp Viral RNA extraction kit. Each disc was transferred to with 560 μl prepared Buffer AVL containing carrier RNA and incubated at room temperature 50° C. for 30 minutes for RNA elution. This was followed by steps as per manufacture instruction (QiaGen). The RNA was eluted in 60 ul RNAase free water. 5 ul of RNA template was used for SARS CoV 2 N1 and N2 assays detection by RTPCR using CFX96 Touch™ real time RT PCR platform (Biorad).

The washed filters were dried at room temperature for 30 minutes and viral RNA eluted by incubating the filter paper discs in 100 μl RNAase free water at 50 C for 30 min. in a water bath.

70% ethanol and 10% to 20% RNAase free water and 10%-20 inactivating solution wherein the inactivating solution is a combination of protein denaturants selected from detergent(s) and chaotropic salt(s), weak base(s), chelator(s) and a reducing agent(s). Suitably wherein the inactivating solution comprises or consists of thiocyanic acid with guanidine (1:1), sodium dodecyl sulphate, octoxinol, trometamol, ethylenediaminetetraacetic acid, and DL-1,4-Dithiothreitol. Suitably in the range 12 to 40% thiocyanic acid with guanidine (1:1), 1 to 3% sodium dodecyl sulphate, 0.5 to 2% octoxinol, 0.5 to 1.5% trometamol, 0.1 to 0.6% ethylenediaminetetraacetic acid, and 0.2 to 0.7% DL-1,4-Dithiothreitol was used to prewash the solid matrix.

Suitably, for RNA recovery temperatures of less than 50 degrees centigrade are advantageous. For DNA recovery temperatures greater than 50 degree centigrade are advantageous. Depending on the downstream application, for example where the presence of both RNA based pathogens like SARS CoV2 virus, influenza virus etc. are being determined as well as DNA based pathogens, for example bacterial pathogens or viral pathogens such as Simian varicella virus, Varicella Zoster Virus or Epstein-Barr virus, the ability to obtain both DNA and RNA from a single sample, such as a dried saliva sample would be advantageous. This would allow for simultaneous testing and thus faster screening of subjects with reduced need to obtain samples.

Washing using at least a first wash solution was performed by incubating each disc for 2 minutes under agitation (400 RPM). Following the washing steps, the filters paper discs were air dried at room temperature for 30 minutes and viral RNA eluted by incubating the discs in 100 μl RNAase free water at 500 C for 60 minutes. In another method, the RNA was recovered using QIAamp Viral RNA extraction kit. Each disc was transferred to with 560 μl prepared Buffer AVL containing carrier RNA and incubated at room temperature 50° C. for 30 minutes for RNA elution. This was followed by steps as per manufacture instruction (QiaGen). The RNA was eluted in 60 ul RNAase free water. 5 μL of RNA template was used for SARS CoV 2 N1 and N2 assays detection by RTPCR using CFX96 Touch™ real time RT PCR platform (Biorad). A Trizol method for RNA recovery as described above can also be used.

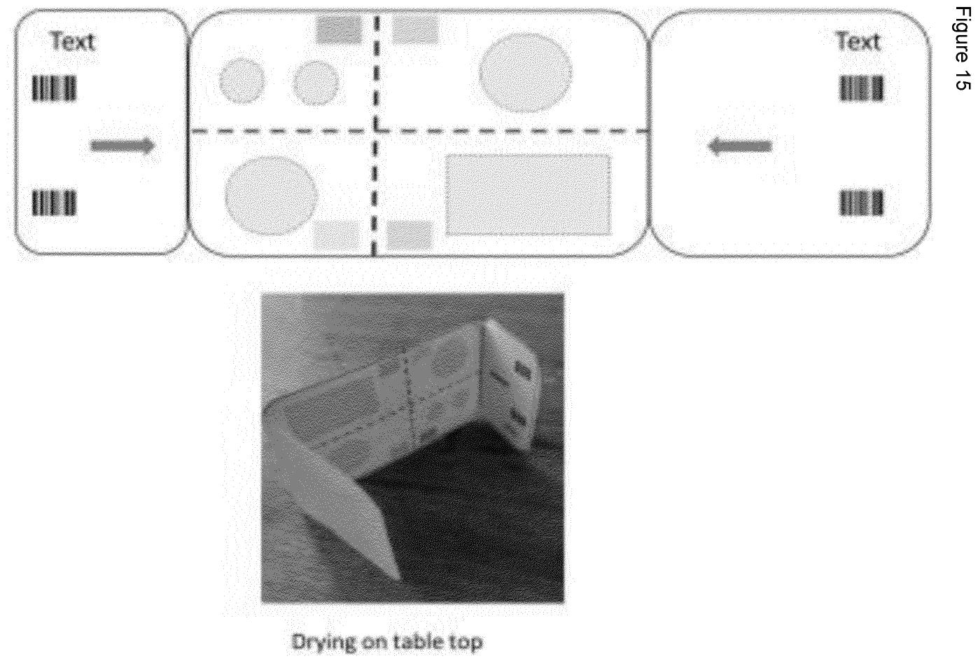

Good amplification curves were observed for the assays tested (FIG. 16). The results are summarized in Table 3, suggesting good analytical performance with regards to amplification efficiency, linearity, repeatability and reproducibility of measurements between different method of RNA extractions, and assays. All measurements were in good agreement and the difference in mean Cq was well within ±1.96 SD.

Advantageously, using the recovery method described herein, both RNA and DNA can be obtained which contrasts only RNA recovery for example when a Trizol method or a method using a QIAamp Viral RNA extraction kit is used.

In the present disclosure, as the prewash buffer contains 10-20% of inactivation solution used to pre-treat the filter paper matrix, suitably contaminants from the pretreated cards such as cell debris, detergents and protein aggregates may be removed in a stepwise manner. The first wash solution removes the salt and other contaminants. Some of these contaminants are known to inhibit PCR reactions and thus it can be advantageous that they are removed before further analysis is undertaken. Suitably the methods of the present disclosure can be undertaken using routine automated plate handlers that are readily commercially available. Further the methods of the present disclosure can be readily automated as they may be undertaken using three components, inactivation solution, ethanol and water.

TABLE 3

| Conditions Tested | TESTING SITE 1 | TESTING SITE 2 | | | |
|---|---|---|---|---|---|
| RNA recovery | Trizol | HemoDx | | QiaAmp | |
| SARS CoV assays | E assay | N1 assay | N2 assay | N1 assay | N2 assays |
| Mastermix | TaqMan ™ Fast | TaqPath ™ | TaqPath ™ | TaqPath ™ | TaqPath ™ |
| tested | Virus 1-Step | 1-Step | 1-Step | 1-Step | 1-Step |
| | Master Mix | Multiplex | Multiplex | Multiplex | Multiplex |
| | (Thermo Fisher) | Master Mix | Master Mix | Master Mix | Master Mix |
| | | (Thermo Fisher) | (Thermo Fisher) | (Thermo Fisher) | (Thermo Fisher) |
| RT PCR | LightCycler ® | CFX96 | CFX96 | CFX96 | CFX96 |
| Platform | 96 System | Touch ™ | Touch ™ | Touch ™ | Touch ™ |
| Tested | (Roche). | system | system | system | system |
| | | (Biorad) | (Biorad) | (Biorad) | (Biorad) |

Analytical Performance Characteristics

| Range in Cq of linear amplification | 24-34 | 19-37 | 19-37 | 19-36 | 19-36 |
|---|---|---|---|---|---|
| Amplification efficiency (ε) | 149.40% | 98.29% | 93.27% | 107.64% | 108.75% |
| Linearity (R²) | 0.964 | 0.9932 | 0.9527 | 0.9956 | 0.9934 |
| Suggested Cq Cut-off | 34 | 37 | 37 | 36 | 36 |
| Limit of Detection (viral copies/ul) | 600 | 60 | 60 | 6 | 6 |
| Repeatibility (% CV) | ++ | +++ | ++ | +++ | +++ |
| Reproducibility | | | | | |
| Lab to Lab | +++ | +++ | | | |
| Assay to Assay | NA | +++ | +++ | +++ | +++ |
| Method to Method | +++ | +++ | | +++ | |
| Agreement between measurements | | | | | |
| (within ± 1.96 SD) | | | | | |
| Lab to Lab | +++ | +++ | | | |
| Assay to Assay | NA | +++ | +++ | +++ | +++ |

| | | | |
|---|---|---|---|
| Method to Method | +++ | +++ | +++ |

+ Good,
++ Very Good,
+++ Excellent

In all cases amplification of target was highly linear and efficient (R2≥0 0.98, and amplification efficiency being >93%). The results obtained were highly repeatable and depended on the assay used. For one assay (N1 assay) the coefficient of variation (CV) observed between replicates was <5%, while for another assay (E-assay) it was 17%, but in all cases less than 20%. Furthermore, the results obtained were highly reproducible irrespective of differences in RNA extraction method, assay type measured, RT PCR instrument used for measurement, testing site facility or operator. There was good agreement between assay-to assay, method-to-method and testing site-to-testing site measurements. All measured data were within ±1.96 SD.

### Example 3—Viral Detection Studies

Clinical details of the nine patients (Sample IDs #73-81) from whom dried saliva samples were collected were considered. For 5 samples (Sample IDs #73-77), NAAT results based on oropharyngeal swabs were collected both pre and post collection dates (Table 4). For two COVID-19 patients, the oropharyngeal (OP) Nucleic Acid Amplification Tests (NAAT) result were negatives (sample ID #73 and 74). These patients were OP swab NAAT positives both 2-3 days pre and 4-5 days post collection dates reflecting the results to be false negatives on the day of collection. Four samples (sample IDs #78-81), the patients were confirmed positives, but no OP swab NAAT results were available on the day of dried saliva collection. However, in case of two samples dried saliva samples were taken from recovering patients 10 days post symptom onset (about 15 days post infection).

TABLE 4

| | | OP Swab NAAT | Dried saliva | | |
|---|---|---|---|---|---|
| | COVID -19 | pre (2-3 days) | collected | | OP Swab |
| | status based | and post | post days | | NAAT |
| Sample | on OP Swab | (4-5 days) | of symptom | | on day |
| # | and Symptoms | sampling date | onset | | of sampling |
| 73 | Positive | Positive | 1 | day | Negative |
| 74 | Positive | Positive | 1 | day | Negative |
| 75 | Positive | Positive | 4 | days | Positive |
| 76 | Positive | Positive | 4 | days | Positive |
| 77 | Positive | Positive | 2 | days | Positive |
| 78 | Positive | NA | NA | | NA |
| 79 | Positive | NA | 10 | days | NA |
| 80 | Positive | NA | 10 | days | NA |

| | | | | |
|---|---|---|---|---|
| 81 | Positive | NA | NA | NA |

For detection of SARS CoV RNA, the nucleocapsid viral proteins N1 and N2 were amplified as viral targets, and human RNase P target amplified as a control to monitor the integrity of the RNA. The detailed RT PCR results is presented in Annexure V. Summarized RT PCR results is presented below (Table 5).

The results suggested a high degree of consistency between dried saliva and gold standard based on swab samples. In samples where NAAT results from oropharyngeal swabs were not available on days of dried saliva sample collection, the dried saliva NAAT were consistent to expected progression of disease where no or low viral titre may be expected in lower respiratory tracts 15 days post infection among recovering patients. In two patients (#73 and #74), the OP NAAT were +ve both two days prior to sample collection and four days after sample collection, while it was −ve on day of dried saliva collection. The samples were collected 1 day post symptom onset. This possibly indicated low viral shedding in lower respiratory tract on the collection day in these patients or the OP swab sampling were not conducted properly. Low viral shedding was confirmed by dried saliva NAAT results of #74 as the Cq for N1 and N2 amplicons were high (32,40 and 34,73 respectively) as compared to other positives. Higher Cq is also observed in case of #78, however no OP swab NAAT is available for the collection date. For one sample (#73), both OP swab and dried saliva NAAT were negative on the day of dried saliva collection, while the patient was confirmed COVID-positive. The NAAT results indicated no or very low viral titre in respiratory tract region on this day possibly indicating some type of early response to infection that may have reduced the viral titre in this region (Table 6).

TABLE 5

| | | | | Covid-19 |
|---|---|---|---|---|
| | | | Cq | Status based |
| Sample | Cq N1 | Cq N2 | RNase | on N1 and |
| # | assay | assay | P assay | N2 assays |
| 73 | — | — | 30.77 | Neg |
| 74 | 32.4 | 34.73 | 27.13 | Pos |
| 75 | 23.08 | 24.04 | 28.43 | Pos |
| 76 | 27.36 | 27.81 | 26.64 | Pos |
| 77 | 31.14 | 31.38 | 31.85 | Pos |
| 78 | 33.74 | 34.88 | 29.58 | Pos |
| 79 | — | — | 30.32 | Neg |
| 80 | — | — | 28.45 | Neg |
| 81 | 28.14 | 28.47 | 31.19 | Pos |
| Internal Positive | 32.16 | 32.98 | — | OK |
| Control (200 cp/ul) | | | | |
| No template | — | — | — | OK |
| Negative Control 1 | — | — | — | OK |
| (Untreated Filter | | | | |
| paper punch) | | | | |
| Negative Control 2 | — | — | 36.77 | Borderline |
| (Untreated Filter | | | | |
| paper punch) | | | | |
| Negative Control | — | — | — | OK |
| 2(Untreated | | | | |
| Filter paper punch) | | | | |

The dried saliva collected using the collection device as described herein can be used as an alternative collection method to nasopharyngeal/oropharyngeal swabs for detecting active SARS-CoV-2 infections. A high degree of consistency of results was observed between dried saliva based on NAAT and swab based NAAT. This also showed stability of the SARS-CoV-2 RNA for more than 6 weeks under ambient conditions using the collection device as discussed herein.

TABLE 6

| | Disease status | Covid 19 status based | Covid-19 | Consistency of Dried saliva results |
|---|---|---|---|---|
| | based on | on OP swab on day of | Status based | to standard of truth based on NAAT |
| Sample # | symptoms | dried saliva collection | on Dried Saliva | and Clinical symptoms |
| 73 | Pos | Neg | Neg | + (consistent with OP Swab on day |
| | | | | of collection) |
| 74 | Pos | Neg | Pos | ++ (consistent with OP Swab NAAT |
| | | | | taken pre and post collection date) |
| 75 | Pos | Pos | Pos | +++ |
| 76 | Pos | Pos | Pos | +++ |
| 77 | Pos | Pos | Pos | +++ |
| 78 | Pos | NA | Pos | +++ |
| 79 | Pos | NA (expected no or low | Neg | ++ (Most probably consistent to OP |
| | | viral titre in respiratory | | swab on this day) |
| | | tract, 15 days post | | |
| | | infection/10 days post | | |
| | | symptom onset) | | |
| 80 | Pos | NA(expected no or low | Neg | ++ (Most probably consistent to OP |
| | | viral titre in respiratory | | swab on this day)++ Most probably |
| | | tract, 15 days post | | YES |
| | | infection/10 days post | | |
| | | symptom onset) | | |
| 81 | Pos | NA | Pos | ++ |

### Example 4—Assay Using Multiple Biofluids

As discussed below, studies were performed with the collection device of the present disclosure (HemoDx device) and a nucleic acid recovery kit for Gut microbiome analysis and also for SARS CoV 2 detection. Multiple biofluids were collected on the same device for the same patient. For example, both saliva and faecal samples can be collected from the patient and the samples analysed to determine the presence or absence of SARS CoV 2.

In this study, the collection device and method as discussed herein were used to extract bacterial gDNA and viral RNA from faeces in combination with the GA-map® Dysbiosis test Lx V2 and GA-map® COVID-19 Faecal Test Kit.

According to the results, it is possible to use the collection device and method for extraction of bacterial gDNA and viral RNA from faeces. However, for acquisition of gDNA, the samples must be diluted initially.

Samples were analysed from a biobank. For each sample three device-cards were prepared. A small amount of sample material was smeared onto filter paper pretreated with Inactivation solution. The cards were stored in room temperature overnight (03:30 PM-07:30 AM). For each of the samples, three 1.5 mL Eppendorf tubes containing two 4.5 mm discs of filter paper were treated according to the extraction protocol, resulting in 100 μL nucleic acid eluate.

Dysbiosis: Three dilutions of gDNA eluate (undiluted, 1:10, 1:100) from four samples, extracted using the method discussed below were analyzed using the GA-map® Dysbiosis test Lx V2. The gDNA from this study (HemoDx) and reference method (GA service lab) was analyzed together on the same plate in order to remove run-to-run variation.

Four samples in three dilutions (undiluted, 1:10, 1:100) were analyzed using the extraction method discussed herein with the GA-map® COVID-19 Faecal Test Kit. Sample 1 and Sample 2 had previously been confirmed Covid-19 positive using the reference method of QIAamp Viral RNA kit (Qiagen) with the GA-map® COVID-19 Faecal Test Kit. There was no reason to suspect that Sample 3 and Sample 4 should be Covid-19 positive.

Extraction protocol used: In the nucleic acid extraction method discussed herein, in this study the filter discs were not transferred to new 1.5 mL Eppendorf tubes after drying in room temperature for 30 minutes. Even so, this does not seem to have affected the results to any great extent.

### Dysbiosis Test Quantification Results:

A quantitative DNA measurement was performed after the 16S rRNA PCR amplification step, using Quant-iT™ on FLUOstar Omega (Table 1). No amplicon was detected in the undiluted samples.

As the undiluted samples did not yield PCR amplicon yields in the expected range, these samples were not included in the downstream analysis. The two other test parameters (1:10 and 1:100 dilution of the eluates) gave yields slightly higher than the reference. Average yields for ref, 1:10 and 1:100 are 30.9, 42.2 and 37.5, respectively.

The dysbiosis index (DI) score was calculated based on probe signals read on Luminex200. The DI index is a scale from 1-5, in which DI 1-2 indicate normal bacterial composition and DI 3-5 indicate a disturbed/dysbiotic microbiota. Inhouse GA bioinformatic tools was used to calculate decimal DI score results.

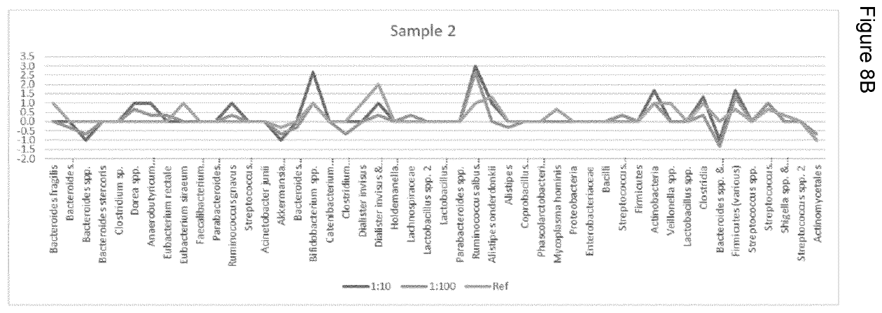

The results (FIG. 7) show overall somewhat higher scores for the present extraction compared to the reference method, but similar replicate variability (STD<0.4 for all three parameters).

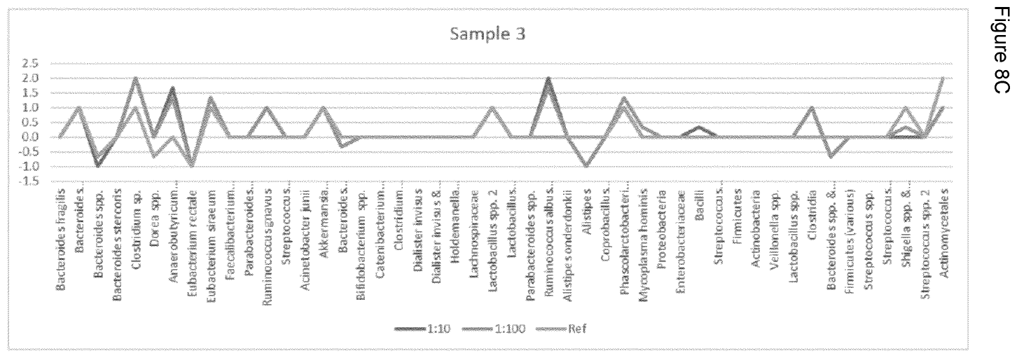

Bacteria Abundance scores: Bacteria Abundance scores were calculated based on probe signals read on Luminex200, using GA-map® Analyzer software. The abundance scores are scores from −3 to +3, in which score 0 indicates normal/expected levels for the bacteria target, −1 to −3 indicate decreased levels and +1 to +3 indicate elevated levels. Inhouse GA bioinformatic tools was also used to interpret the results (FIG. 8).

The results show similar bacteria profiles across the extraction methods but indicate some systematic differences between the reference extraction and that of the present method.
Systematic reduction with extracts of present invention compared to reference: Bacteroides spp. & Prevotella spp., Dialister invisus & Megasphaera micronuciformis, Alistipes
Systematic increase with extracts of present invention compared to reference: Ruminococcus albus & R. bromii, Dorea spp, Anaerobutyricum hallii

In both plates, the negative and positive controls yield a signal (Cq and PFU values) in the expected range. The RP Ct values are above the upper limit for all sample replicates in the 1:100 dilution. The results from these samples are therefore not analyzed further. Moreover, the RP Ct values are above the upper limit for ≥1 replicates for all samples in all parameters. Of the included samples, two were known Covid-19 positive (S1 and S2) and two Covid-19 negative (S3 and S4). Samples S1 and S2 have expected N1 and N2 Ct values 27 and 31, respectively, and an expected RP Ct value of 32 (results from original analysis using the GA-map® COVID-19 Faecal Test in May 2020; data not shown).

The Covid-19 samples were analyzed using a CFX96 C1000 real-time thermal cycler, and the resulting file was prepared using Bio-Rad CFX Maestro Software.

| Undil. | Sample 1 | Sample 2 | Sample 3 | Sample 4 |
|---|---|---|---|---|
| N1 | 32.5 | 34.3 | NA | NA |
| N2 | 32.4 | 35.6 | NA | NA |
| RP | 36.0 | 38.2 | 36.5 | 36.2 |

| 1:10 | Sample 1 | Sample 2 | Sample 3 | Sample 4 |
|---|---|---|---|---|
| N1 | 32.0 | 34.9 | NA | NA |
| N2 | 33.1 | 37.5 | NA | NA |
| RP | 37.9 | 37.4 | 36.5 | 39.4 |

The results show positive Covid-19 signal in samples S1 and S2 and negative in S3 and S4, as expected. However, the signals are significantly lower for all three target sequences (N1, N2 and RP) compared to the original analysis of the same samples, indicating decreased sensitivity compared to GA-map® COVID-19 Faecal Test.

The collection of multiple different types of biofluids, for example from different sites of a patient can be advantageous as for example, during initial days (1-8 days post infection) the virus is present in oral region and can be detected using saliva or swabs while during latter stages of infection (>8 days, long Covid) the virus can be found in stool samples but not in saliva samples or swab samples (RTPCR negative). Suitably a device that is capable of being used to collect different types of samples and to stabilise these for transport to allow testing provides advantageous flexibility of use of the collection device

Moreover the collection device, for example same card frame, may be used to collect different biospecimens from the same patient, for example both saliva and stool samples are collected for SARs CoV 2 such that a determination of the stage of the disease, early if present in saliva, late when present in stool samples can be undertaken and if suitable detection of both RNA and DNA may be undertaken using the same device.

Alternatively, the collection device may be used to collect and stabilise biological samples such that RNA recovered from the collection device can be used for gene expression profiling and microRNA profiling, while DNA recovered from the collection device can be used for genotyping purposes or microbiome analysis. Saliva, blood or faecal samples can be obtained as per required. As will be appreciated, nucleic acid obtained using the collection device discussed herein might be used for various clinical applications, such as diagnosing diseases, selecting therapy, and determining disease severity for example of infectious diseases, different cancer types (breast, prostate, pancreas, lung, colon etc.) and neurodegenerative diseases such as Alzheimer's disease and other memory disorders, Parkinson disease, Huntington's disease, motor neuron disease, multiple system atrophy and progressive supranuclear palsy.

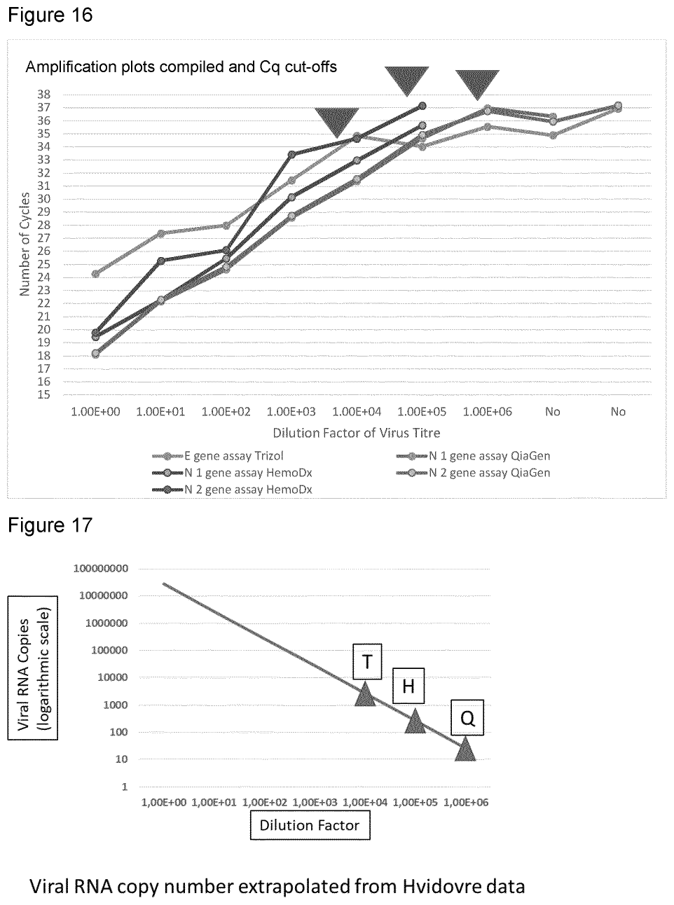

As indicated in FIGS. 16 and 17, following extraction using the method of the present invention, template formation is at least comparative with other conventional methods of extraction as used in the art.

### Example 5—Large-Scale Double-Blind Viral Testing

As an extension to the assay of Example 3 a further study was conducted. This used blind testing to demonstrate the use of self-sampling using collection devices of the present invention to determine the presence or absence of SARS CoV 2.

The study was performed by an independent contract research organisation and comprised 100 COVID-19 (SARS CoV-2) positive and 100 COVID-19 (SARS CoV-2) negative subjects.

As controls, nasopharyngeal and oropharyngeal swab samples were obtained per ICMR recommendations by healthcare professionals (standard of truth). Next, patients were provided with a kit of the fifth aspect (HemoDx kit) and instructed to provide saliva samples, either self-sampled when in the hospital setting on the same day, or self-sampled at home on the same day and post the collection device under ambient conditions to the study's postal address.

Participants were instructed to provide at least two drops of saliva to the designated area on the collection device, then allow the saliva to dry under ambient conditions for at least 45 minutes before sealing it in the relevant bag (which contained desiccant).

All samples were blinded by removing patient identification and assigning unique barcodes to each study sample. Blinding was not performed by the same person who performed the subsequent laboratory analysis.

The presence or absence of SARS CoV-2 (as well as copy number) was determined using RT PCR-based detection of SARS CoV-2 RNA, using the ORF 1 ab and N genes. ARIDIA COVID-19 was used as a positive control, ARIDIA dH₂O was used as a negative control.

The nasopharyngeal and oropharyngeal swab samples were tested using the Covipath COVID-19 RT-PCR Kit from Thermofisher Scientific. These kits are designed for the qualitative detection of ORF 1 ab and N genes of the SARS-CoV-2 genome by real time Reserve Transcriptase PCR.

RNA was collected from the saliva samples collected using collection devices of the present invention using a QiaGen viral RNA kit, following the manufacturer's instructions. However, other RNA collection kits are available.

The RNA recovered from the saliva samples collected using collection devices of the present invention were assessed for the presence or absence of SARS CoV-2 as follows:
1. The necessary reagents, including a Positive Control were thawed on ice.
2. One PCR master mix was prepared per primer/probe pair: see Table 7.
3. 20 μL of the RT-PCR master mix was dispensed onto a 96-well PCR plate according to the sample setup.
4. 5 μL of template was added to the plate according to the plate setup.
   a. Positive control: ARIDIA COVID-19 (PosCtr)
   b. Negative control: ARIDIA dH₂O (NTC)
5. The plate was sealed and loaded the samples on a IANLONG-GENTIER 48E RT PCR platform. The PCR program is given in Table 8.

TABLE 7

| Component | μL per well |
|---|---|
| Master A | 0.8 |
| Master B | 2.0 |
| Buffer | 4.0 |
| Primer - Probe Mix | 3.0 |
| Molecular Biology Grade Water (MBGW) | 5.2 |
| Master Mix Total | 15.0 |
| Positive Control/Sample RNA/Negative Control | 5.0 |
| Total volume per reaction | 20.0 |

TABLE 8

| Cycles | Step | Time | | Temp (° C.) |
|---|---|---|---|---|
| 1 | RT Incubation | 15 | min | 50 |
| 1 | Initial Denaturation | 2.5 | min | 95 |
| 40 | Denaturation | 15 | s | 94 |
| | Annealing (Data Collection) | 40 | s | 55 |

### Conclusion:

A total of 225 participants participated in the trial, however 15 participants were pre-screen failure and 10 were screen failed—exclusion criteria were that the patients must be over 18 years of age and would provide signed written informed consent.

Out of the remaining participants, 200 enrolled to detect Covid-19 using dried saliva spots. 100 patients were Covid-positive and 100 patients Covid-negative.

For 3 of the patients, saliva samples were collected 5 days after the nasopharyngeal and oropharyngeal swabs. For 5 of the patients, saliva samples were collected 6 days after the nasopharyngeal and oropharyngeal swabs.

For 7 of the samples, results based on nasopharyngeal and oropharyngeal swab (RTPCR) were positive for Covid-19. However, the corresponding saliva samples using the device of the present invention provided a negative result. Two days after initial sample collection, these patients were tested again using collection devices of the present invention and two tested positive, five tested negative.

During the study there were no adverse effects reported, apart from the COVID infection for the participants.

### Sensitivity:

### RT PCR Detection Based on “N” Gene:

Nasopharyngeal swabs (gold standard): 96%
Dried Saliva using a collection device of the present invention: ≥95%.

### RT PCR Detection Based on “ORF” Gene:

Nasopharyngeal swabs (gold standard): 90%
Dried Saliva using a collection device of the present invention: ≥95%.

Overall, there was a 97% consistency in results observed in results observed between dried saliva collected using a collection device of the present invention and the nasopharyngeal and oropharyngeal swabs.

This demonstrates that sampling saliva using a collection device of the present invention is as reliable/more reliable than nasopharyngeal swabs depending on the assays used for RT PCR detection for COVID-19.

### Specificity:

### Test Specificity Based on RT PCR Detection:

Nasopharyngeal swabs (gold standard): 100%
Dried Saliva using a collection device of the present invention: 100%

100% compliance between Swab samples and HemoDx Dried Saliva Collection Device, and 100% compliance to Clinical Diagnosis—i.e. the results of all healthy controls were negative using swab samples and the collection devices of the present invention.

In summary, the detection of SARS CoV 2 using saliva samples collected using collection devices of the present invention is as good/better than the current gold standard with buccal swabs (specificity=100%, sensitivity=≥95%).

### Example 6—Expanded Viral Testing

As an extension to the assay of Example 3, a further study was designed to assay whether a collection device of the present invention could be used to detect the presence or absence of other respiratory viruses such as influenza A, influenza B and respiratory syncytial virus A/B in dried saliva.

This is proposed to be an equivalence study, comparing:
a) the RT PCR based detection of respiratory viruses (other than SARS CoV2) using nasopharyngeal/oropharyngeal swabs (Standard of truth) to
b) the RT PCR based detection of respiratory viruses using dried saliva samples collected using a collection device of the present invention.

Biofluid samples would be taken from at least 30 male or female subjects on the same day, using both nasopharyngeal/oropharyngeal swabs and collection devices of the present invention. For collection devices of the present invention, saliva is to be the sampled biofluid.

These samples would then be allowed to dry under ambient conditions for 45 minutes before transport to the analysis site. For collection devices of the present invention, the saliva samples could be taken by the study participants at home, who would then post the samples to be studied.

Where a subject's nasopharyngeal/oropharyngeal swab tested positive via RT PCR for influenza A, influenza B or respiratory syncytial virus A/B (at least 10 subjects per condition), the corresponding collection device of the present invention would be assayed for the presence or absence of that virus using RT PCR.

Without wishing to be bound by theory, it is believed that the collection devices of the present invention will provide equivalent or improved accuracy when compared with the samples obtained via nasopharyngeal/oropharyngeal swab. As such, the collection

### Example 7—Testing for Alzheimer's Disease (AD) and Parkinson's Disease (PD) Using Faecal Samples

Mutations on the two homologous presenilin genes: presenilin 1 (PS1, MIM 104 311) located on chromosome 14, and presenilin 2 (PS2, MIM 600 759) located on chromosome 1, are most common and are responsible for over half of the known familial Alzheimer's disease (AD). Mutations in the gene for amyloid precursor protein (APP, MIM 104 760) located on chromosome 21) are comparatively less. In addition the E4 allele of the ApoE is associated with the sporadic forms of AD. Along with positive family history, an early onset (in the 40s and 50s) which is common to all monogenic forms, testing for the presence of such a mutation in a subject should act as an indication for molecular genetic diagnosis. Suitably the DNA can be isolated from biofluids collected on the collection device, such as blood, and tested for the presence or absence of these mutations.

Parkinson disease (PD) is inherited in a Mendelian autosomal dominant or autosomal recessive fashion in a small number of families. Mutations were found in α-synuclein (SNCA) and leucine-rich repeat kinase2 (LRRK2) genes for late-onset disease and parkin (PARK2), ubiquitin carboxy-terminal hydrolase L1 (UCH-L1), PTEN Induced Putative Kinase1 (PINK1), oncogene DJ1 (DJ1) for early onset. Point mutations, duplications, and triplication in the α-synuclein gene, which is located on chromosome 4, are a characteristic of PD and they occur in most forms including the rare early onset familial form of PD. Genes and gene products have been identified by characterizing the monogenetic autosomal dominant forms of PD. Several gene products of the mutated genes in the autosomal dominant forms have been linked to mitochondrial dysfunction, oxidative stress, and mishandling of impaired or aberrant forms of the gene products (e.g., oligometric α-synuclein). More than 70 mutations on the large parkin gene have been associated with the early-onset form of Parkinsonism. Mutations in the parkin gene may account for PD in as many as 50% of familial cases of autosomal recessive juvenile Parkinsonism. Another gene ubiquitin carboxy-terminal hydrolase L1 (UCH-L1) located on chromosome 4 encodes a protein which belongs to the family of deubiquitinating enzymes. Protein UCH-L1 constitutes 1% of brain protein and its function is presumed to act to recycle ubiquitin by hydrolyzing the ubiquitinated peptides. This enzyme plays a role in modifying the damaged proteins that might otherwise accumulate to toxic levels in the neuron. Also two homozygous mutations in PINK1 gene associated with PD were found in Spanish and Italian families. Evidence suggests DJ-1 protein involvement in oxidative stress and neurodegeneration. Slow progression of symptoms with sustained response to levodopa treatment is the clinical characteristics of DJ-1 Parkinsonism. It is considered that determination of the genetic status of subjects may promote an understanding of the mechanisms of brain neuronal maintenance.

miRNAs belong to a family of short, single-stranded 21-22 nucleotides-long non-coding RNAs that constitute about 1% of all human genes. They represent the most abundant class of small RNAs in animals. Altered expression of microRNAs (miRNAs) in many disease states, including neurodegeneration along with applications of miRNAs in biological fluids in different pathologies make them promising candidates as neurodegenerative disease biomarkers that may lead to identify new therapeutic targets.

Plasma miRNA biomarkers were reported to detect MCI, where an initial pool of miRNAs was selected among known brain- and neuron-enriched miRNAs. The researchers then identified biomolecular diagnostic marker pairs represented by two sets: the “miR-132 family” that consist of miR-128/miR-491-5p, miR-132/miR-491-5p and miR-874/miR-491-5p and the “miR-134 family” comprising miR-134/miR-370, miR-323-3p/miR-370 and miR-382/miR-370 with fairly high sensitivity and specificity at 79-100% and 79-95%, respectively. In a separate longitudinal study, the identified miRNA biomolecular diagnostic marker pairs successfully detected MCI in majority of patients at asymptomatic stage 1-5 years prior to clinical diagnosis.

A study using qRT-PCR suggested that in peripheral blood the expression levels of miR-1, miR-22-5p, and miR-29 allow to distinguish PD patients from healthy subjects, and also miR-16-2-3p, miR-26a-2-3p, and miR30a differentiate between treated and untreated patients. In a recent study using next generation sequencing for total blood leukocytes it was found that, 16 miRNAs including miR-16, miE-20a and miR-320 significantly altered in PD patients compared to healthy controls.

Several studies have indicated that early detection of Alzheimer's disease and Parkinson's disease is possible using blood gene expression data. Transcriptomics typically suffers from the requirement of extreme cold chain logistics (dry ice) to maintain the integrity of mRNA. Advantageously the present device and methods mitigates the need for such cold chain logistics.

Features of the intestinal microbiome have been linked to particular disorders and diseases, including neurological diseases (Alzheimer's and Parkinson's disease For example, sequencing of intestinal microbiota has revealed that the relative abundance of Enterobacteriaceae in the faeces of PD patients is strongly correlated with the severity of postural instability and gait difficulties compared to controls. Several studies have shown correlation changes between the salivary and faecal microbiotas which highlight the possibility to use saliva-based screening as a substitute to or in addition to faecal samples in microbiologic studies of systemic diseases.

### Example 8—Testing for Alzheimer's Disease (AD) Using Peptide Biomarkers/Analytes of Interest

A feasibility study was performed, detecting the presence and concentration of peptide fragments Aβ-40 and Aβ-34 (involved in the amyloid cascade) in the saliva of healthy patients. This was performed to assess the feasibility of detecting the presence and concentration of peptide fragments Aβ-40 and Aβ-34 in patients as an indicator for Alzheimer's disease.

The peptide fragments were collected and stored for subsequent analysis using the method of the third aspect of the present invention, namely:
1. 100 μL saliva spotted on a collection device of the present invention, wherein the inactivation solution comprised 0.45% CHAPS and 0.1% thioflavin. The average diameter of the spots were approximately 20 mm.
2. The saliva was allowed to dry at RTP for ˜45 minutes before being sent through the postal service to a lab for analysis.
3. Five 6 mm diameter discs of the solid matrix containing the dried saliva were punched from the collection device, to which was added 200 μL of 1×PBS buffer at pH 7.4, containing 0.45% CHAPS and 0.1% Thioflavin.
4. This was briefly vortexed, microcentrifuged and incubated at room temperature for 5 minutes, with shaking at 5500 rpm in a tabletop shaker.
5. The supernatant was then aspirated away.
6. 150 μL of elution buffer was then added to the solid matrices, wherein the elution buffer comprised:

| Item | Concentration |
|---|---|
| PBS at pH 7.4 | x1 |
| EDTA | 0.30% |
| BSA | 0.10% |
| Proclin | 0.05% |
| Thioflavin S | 0.10% |
| Protease Inhibitors | 2% |
| CHAPS | 0.05% |

7. This mixture was then incubated at 37° C. for 1 hr, with shaking at 5500 rpm in a tabletop shaker.
8. The mixture was then briefly vortexed and microcentrifuged prior to the supernatant being aspirated to provide a solution comprising the analyte of interest.
9. This was then stored at −20° C. until it was analysed by immuno-detection.

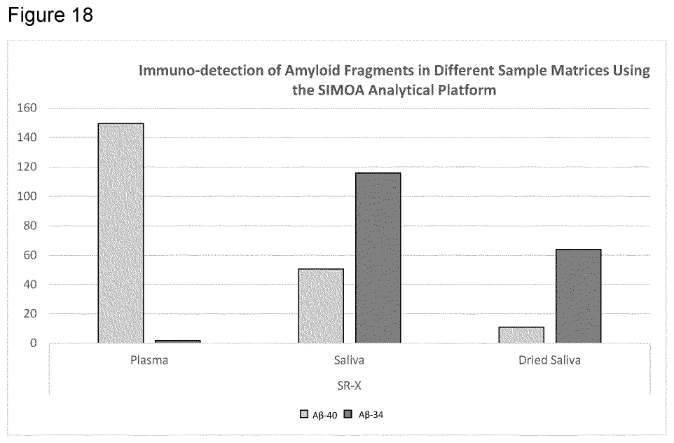

The concentration of Aβ-40 and Aβ-34 from the saliva samples were measured using SIMOA (SR-X), as shown in FIG. 18. As mentioned, these results were obtained from saliva obtained from healthy control subjects. As the concentration of Aβ-40 and Aβ-34 in saliva increases in patients affected by Alzheimer's is elevated compared to healthy controls, this is considered sufficient for the purposes of demonstrating feasibility of the present invention.

Surprisingly, these peptide fragments—dried onto collection devices of the present invention—could be detected for at least six weeks after they were collected, when the collection devices were stored under ambient conditions.

Suitably as the collection device and method of the present invention allows collection of biofluids and stabilization of analytes such that both DNA and RNA may be analysed, the collection device and method may be advantageous in determining genotypes using gene based methods and RNA, for example miRNA or mRNA for large scale miRNA or gene expression profiling for diagnosing or monitoring diseases, selecting therapy, and determining disease severity.

Furthermore, it will be obvious to person skilled in the art that the collection device and kit to collect biofluids, as described herein, can also be used to collect other fluid types such as wastewater or sewage and used to track or detect the presence or absence of bacterial or viral pathogens. For example, it can be used to monitor and early detection of SARS CoV2 in wastewater or sewage as the virus is shed in faeces by infected individuals and can be measured in wastewater allowing for community or population level surveillance of pathogen spread in an area.

1. A collection device for the collection and storage of an analyte of interest from a biofluid sample,
wherein the biofluid sample is suspected of comprising the analyte of interest and comprises one or more pathogens,
wherein the collection device comprises a solid matrix incorporating an inactivating solution adapted to inactivate substantially all of the one or more pathogens present in the biofluid sample and preserve at least a portion of the analyte of interest in a format that permits subsequent analysis,
wherein the inactivating solution comprises a combination of protein denaturants selected from one or more detergents, one or more chaotropic salts, one or more weak bases, one or more chelators and one or more reducing agents, and
wherein in use, when the biofluid sample is provided to the solid matrix, substantially all of the one or more pathogens in the biofluid sample is inactivated and at least a portion of any analyte of interest present in the biofluid sample is preserved in a format that permits subsequent analysis.

2. The collection device of claim 1, wherein the inactivation solution is adapted to inactivate substantially all of the one or more pathogens within 45 minutes of the biofluid sample being provided to the collection device.

3. The collection device of claim 1, wherein the inactivation solution is adapted to inactivate substantially all of the one or more pathogens present in the biofluid sample such that at least 90%, at least 95%, at least 99%, at least 99.9 or at least 99.99% of the infective micro-organisms present in the one or more pathogens are inactivated.

4. The collection device of claim 3, wherein the inactivation solution is adapted to inactivate substantially all of the one or more pathogens present in the biofluid sample such that no infective micro-organisms can be detected.

5. The collection device of claim 1, wherein the inactivation solution is adapted to inactivate substantially all of the one or more pathogens when the one or more pathogens are at a high titre.

6. The collection device of claim 1, wherein the solid matrix is a filter paper.

7. The collection device of claim 1, wherein the inactivating solution comprises 12-40 wt % of the chaotropic salts.

8. The collection device of claim 1, wherein one or more of the chaotropic salts are selected from a group consisting guanidium salts such as guanidium isothiocyanate, guanidine thiocyanate or guanidine hydrochloride, sodium iodide, sodium perchlorate, sodium thiocyanate and potassium iodide.

9. The collection device of claim 1, wherein one or more of the detergents are selected from a group consisting ionic detergents such as sodium dodecyl sulphate, deoxycholate, cholate and sarkosyl, and non ionic detergents such as the Triton family, i.e. octoxinol, such as Triton X100 or Triton X-114, Nonidet P-40, Igepal® CA-630 and the Tween family such as Tween-20 or Tween-80.

10. The collection device of claim 1, wherein one or more of the weak bases are selected from a group consisting 2-Amino 2-hydroxymethyl-propane-1,3-diol, 2-(N-morpholino) ethanesulfonic acid, 3-(N-morpholino)propane sulfonic acid, citrate buffers, 4-(2-hydroxyethyl)-1-piperazineethanesulfonic acid, and phosphate buffers.

11. The collection device of claim 1, wherein one or more of the chelators are selected from a group consisting ethylenediaminetetraacetic acid, Ethyleneglycoltetraacetic acid and 8-Hydroxyquinoline.

12. The collection device of claim 1, wherein one or more of the reducing agents are selected from a group consisting dithiothreitol, dithioerythritol, L-glutathione, 2-carboxyethyl phosphine hydrochloride and 2-mercaptoethanol.

13. The collection device of claim 1, wherein the inactivating solution comprises guanidine isothocyanate, sodium dodecyl sulphate, octoxinol, Tris base, ethylenediaminetetraacetic acid, and dithiothreitol.

14. The collection device of claim 1 comprising at least 50 wt % cellulose, and an inactivating solution in the range 12 to 40 wt % guanidine isothiocyanate, 1 to 3 wt % sodium dodecyl sulphate, 0.5 to 2 wt % octoxinol, 0.5 to 1.5 wt % Tris base, 0.1 to 0.6 wt % ethylenediaminetetraacetic acid, and 0.2 to 0.7 wt % dithiothreitol.

15. The collection device of claim 1, wherein the analyte of interest is a nucleic acid or a protein/peptide.

16. The collection device of claim 1, wherein the analyte of interest is a component of at least one of the pathogens, and wherein the at least one of the pathogens is a viral agent selected from corona virus, influenza virus, norovirus, rabies (lyssavirus), Human papillomavirus, Epstein-Barr virus, Herpes simplex virus, Hepatitis virus, in particular Hepatitis C virus, Monkeypox virus and HIV.

17. The collection device of claim 1, wherein the analyte of interest is a component of at least one of the pathogens, and wherein the at least one of the pathogens is a bacterial agent related to microbiome profiling, bacterial dysbiosis, periodontitis, dental carries, diabetes, obesity, metabolic disorder, cancer, CVD, immuno-related systemic diseases.

18. The collection device of claim 1, wherein the analyte of interest is a biomarker for a neurodegenerative disease, in particular Alzheimer's disease or Parkinson's disease.

19. The collection device of claim 1, wherein the inactivating solution is able to inactivate substantially all SARS CoV 2 virions present in the biofluid sample, with no detectable virions within 45 minutes of providing the biofluid sample to the collection device.

20. The collection device of claim 1, wherein the biofluid sample is a saliva sample.

21. A method for detecting an analyte of interest provided from a dried biofluid, from a collection device, wherein the analyte of interest is a nucleic acid,
wherein the collection device comprises a solid matrix that was pre-treated with an inactivating solution adapted to inactivate substantially all of the one or more pathogens present in the biofluid sample and preserve at least a portion of the analyte of interest in a format that permits subsequent analysis,
wherein the inactivating solution comprises a combination of protein denaturants selected from one or more detergents, one or more chaotropic salts, one or more weak bases, one or more chelators and one or more reducing agents, and
wherein the method comprises the steps:
a. washing a portion of the solid matrix comprising the dried biofluid with prewash buffer at room temperature to form a previously dried biofluid, wherein the prewash buffer comprises:
i. 60%-80% ethanol, and
ii. inactivating solution comprising a combination of protein denaturants selected from one or more detergents, one or more chaotropic salts, one or more weak bases, one or more chelators and one or more reducing agents,
b. washing a portion of the solid matrix comprising the previously dried biofluid with a first wash solution, wherein the first wash solution comprises at least 70% ethanol, and
c. eluting the analyte of interest from the solid matrix by incubating the solid matrix RNAase free water.

22. The method of claim 21, further comprising washing a portion of the solid matrix comprising the previously dried biofluid with a second wash solution at room temperature, wherein the second wash solution comprises at least 95% ethanol.

23. The method of claim 21, wherein the step of eluting the analyte of interest provides both RNA and DNA from the dried biofluid.

24. The method of claim 21, wherein the dried biofluid is saliva or faecal matter.

25. A method for detecting an analyte of interest provided from a dried biofluid, from a collection device, wherein the analyte of interest is a protein, a polypeptide, an oligopeptide or a peptide,
wherein the collection device comprises a solid matrix that was pre-treated with an inactivating solution adapted to inactivate substantially all of the one or more pathogens present in the biofluid sample and preserve at least a portion of the analyte of interest in a format that permits subsequent analysis,
wherein the inactivating solution comprises a combination of protein denaturants selected from one or more detergents, one or more chaotropic salts, one or more weak bases, one or more chelators and one or more reducing agents, and
wherein the method comprises the steps:
eluting the analyte of interest from the solid matrix by incubating at least a portion of the solid matrix comprising the dried biofluid with an elution buffer, then removing the elution buffer, wherein the elution buffer comprises:
one or more buffering salts,
one or more solubilisation agents and
one or more protease inhibitors.

26. The method of claim 25, wherein the elution buffer comprises
One or more buffering salts to provide a pH of 6-9,
0.1-0.6% of the one or more solubilisation agents, and
1-3% of the one or more protease inhibitors.

27. The method of claim 25, wherein the elution buffer comprises one or more of the following:
One or more chelating agents,
One or more carrier proteins,
One or more phosphatase inhibitors, and
One or more biocides.

28. The method of claim 27, wherein the one or more chelating agents are selected from a group consisting ethylenediaminetetraacetic acid (EDTA), Ethyleneglycoltetraacetic acid (EGTA) and 8-Hydroxyquinoline (8HQ).

29. The method of claim 27, wherein the one or more carrier proteins are be selected from a group consisting bovine serum albumen (BSA) and keyhold limpet hemocyanin (KLA).

30. The method of claim 27, wherein the one or more biocides are selected from a group consisting ProClin and sodium azide.

31. The method of claim 27, wherein the elution buffer comprises:
one or more buffering salts to provide a pH of ˜7.4,
0.1-0.6% of the one or more solubilisation agents,
1-3% of the one or more protease inhibitors,
0.3-1.5% of the one or more chelating agents,
0.05-2% of the one or more carrier proteins and
0.03-0.07% of the one or more biocides.

32. The method of claim 27, wherein the elution buffer comprises:
PBS to provide a pH of 7.4
0.01-0.1% of one or more non-denaturing zwitterionic detergents,
0.1-0.5% Thioflavin,
1-3% of the one or more protease inhibitors,
0.3-1.5% of the one or more chelating agents,
0.05-2% of the one or more carrier proteins,
0.03-0.07% of the one or more biocides.

33. The method of claim 27, wherein the elution buffer comprises:
PBS to provide a pH of 7.4,
0.01-0.1% CHAPS,
0.1-0.5% Thioflavin S,
1-3% protease inhibitors,
0.3-1.5% ethylenediaminetetraacetic acid,
0.05-2% BSA,
0.03-0.07% ProClin.

34. The method of claim 27, wherein the method further comprises washing the solid matrix with a pre-elution buffer, prior to eluting the analyte of interest from the solid matrix, wherein the pre-elution buffer comprises one or more buffering salts, one or more solubilisation agents and one or more protease inhibitors.

35. A method for detecting an analyte of interest provided from a dried biofluid, from a collection device, where the analyte of interest is a protein, a polypeptide, an oligopeptide or a peptide,
wherein the collection device comprises a solid matrix that was pre-treated with an inactivating solution adapted to preserve at least a portion of the analyte of interest in a format that permits subsequent analysis,
wherein the inactivating solution comprises one or more protease inhibitors and one or more solubilising agents and
wherein the method comprises the steps:
eluting the analyte of interest from the solid matrix by incubating at least a portion of the solid matrix comprising the dried biofluid with an elution buffer, then removing the elution buffer, wherein the elution buffer comprises
one or more buffering salts,
one or more solubilisation agents and
one or more protease inhibitors.

36. A kit comprising a collection device of claim 1 and at least one of
a. a pipettor to drop the biofluid sample onto the collection device,
b. a biofluid sample collection cup,
c. a biohazard marked bag,
d. desiccant, and
e. a return shipping envelope for storage and transport of biofluid samples to a designated laboratory for analysis.

37. A method of using the collection device of claim 1 to undertake at least one of profiling the microbiome, genotyping purposes, diagnosing diseases, selecting therapy, and determining disease severity.

38. A method of using the collection device of claim 1 with the method of a) any of claims 21-24, b) any of claims 25-34 or c) claim 35 to undertake at least one of profiling the microbiome, genotyping purposes, diagnosing diseases, selecting therapy, and determining disease severity.

39. The collection device of claim 1 to detect neurodegenerative diseases, including Alzheimer disease and Parkinson disease, configured to perform the method of:
a. washing a portion of the solid matrix comprising the dried biofluid with prewash buffer at room temperature to form a previously dried biofluid, wherein the prewash buffer comprises:
i. 60%-80% ethanol, and
ii. inactivating solution comprising a combination of protein denaturants selected from one or more detergents, one or more chaotropic salts, one or more weak bases, one or more chelators and one or more reducing agents,
b. washing a portion of the solid matrix comprising the previously dried biofluid with a first wash solution, wherein the first wash solution comprises at least 70% ethanol, and
c. eluting the analyte of interest from the solid matrix by incubating the solid matrix RNAase free water.


SEQUENCE LISTING (3 sequences)

SEQ ID NO: 1 - DNA, 26 bp, synthetic construct
  source 1..26: mol_type = other DNA
acaggtacgttaatagttaatagcgt

SEQ ID NO: 2 - DNA, 22 bp, synthetic construct
  source 1..22: mol_type = other DNA
atattgcagcagtacgcacaca

SEQ ID NO: 3 - DNA, 26 bp, synthetic construct
  source 1..26: mol_type = other DNA
  modified_base 1: mod_base = OTHER; note = This residue is modified with a 6-fluorescein amidite (FAM) at the 5' site.
  modified_base 26: mod_base = OTHER; note = This residue is modified with a Black Hole Quencher-1 (BHQ-1) at the 3' site.
acactagccatccttactgcgcttcg